(19) United States
(12) Patent Application Publication
(10) Pub. No.: US 2020/0359887 A1
(43) Pub. Date: Nov. 19, 2020

(54) OCT RADIOMIC FEATURES FOR DIFFERENTIATION OF EARLY MALIGNANT MELANOMA FROM BENIGN NEVUS

(71) Applicant: Wayne State University, Detroit, MI (US)

(72) Inventors: Kamran Avanaki, Ann Arbor, MI (US); Peter Andersen, Roskilde (DK)

(21) Appl. No.: 15/931,937

(22) Filed: May 14, 2020

Related U.S. Application Data
(60) Provisional application No. 62/847,391, filed on May 14, 2019.

(51) Int. Cl. A61B 3/10; A61B 3/00; A61B 3/12; G01N 21/47; G01N 21/64

(52) U.S. Cl. CPC A61B 3/102; A61B 3/0025; G01N 2021/1787; G01N 21/4795; G01N 21/6456; A61B 3/1225

(57) ABSTRACT

A system and method of optical coherence tomography includes defining a suspect region-of-interest (SROI) for a suspect lesion in a first OCT B-scan image, defining a healthy region-of-interest (HROI) near the suspect lesion in a second OCT B-scan image, extracting optical properties from the SROI and from the HROI, obtaining an averaged A-line in the SROI and in the HROI, creating a set of normalized optical radiomic features from the averaged A-line in the SROI and in the HROI, and evaluating the set of normalized optical radiomic features to distinguish whether the suspect lesion is consistent with melanoma.

### CROSS-REFERENCE TO RELATED APPLICATIONS

This application in based on and claims priority to U.S. Provisional Patent Application No. 62/847,391 filed on May 14, 2019, which is hereby incorporated by reference in its entirety.

### TECHNICAL FIELD

This disclosure relates generally to a system, apparatus and method of identifying, detecting and/or diagnosing cancer including, for example, melanoma by differentiation of early malignant melanoma from benign nevus.

### BACKGROUND

Melanoma is an increasingly important public health problem worldwide. The incidence of melanoma has been rising faster than any other cancer, e.g., due to changes in sun exposure behavior as well as climate change. Melanoma was responsible for over 50,000 deaths per year with an age-standardized rate of one death per 100.000 persons.

Healthy and non-healthy tissues can be well differentiated based on their characteristics. There are characteristic differences in number, size and distribution of melanocytes seen in healthy skin, nevi and melanoma. In healthy skin, melanocytes occur singly along the basal layer of the epidermis at a rate of approximately 1 for every 10 keratinocytes. In benign nevi, there is an increase in the number of melanocytes and they occur grouped into nests, but they maintain their normal size. In melanoma, there is an increase in the number of melanocytes and the cells are larger and atypical. The atypical melanocytes are frequently seen in layers of the epidermis above the basal layer, known as pagetoid spreading.

Traditionally the process of diagnosing a melanoma begins with visual inspection of skin lesions. Visual evaluation criteria for suspected melanomas include the ‘ABCDE’ criteria (Asymmetry, Border irregularity, Color variation, Diameter >6 mm, Evolving). Skin lesions that fulfill the ABCDE criteria for melanoma are then biopsied for histopathologic analysis. The specificity (˜59% to 78%) of visual inspection criteria varies widely based on the experience of the clinician and when used singly or in combination. This wide variability in specificity is due to both subjective interpretation by physicians as well variability in the number of criteria present in a given suspicious lesion. This can result in unnecessary biopsy of benign lesions, ranging for example from 15-30 benign lesions biopsied to diagnose one melanoma. Performing a biopsy can result in pain, anxiety, scarring and disfigurement for patients, as well as a cost for the healthcare system. Another challenge is finding the correct lesion(s) to biopsy in a patient with many pigmented lesions. Toward addressing these challenges, several imaging techniques have been developed to noninvasively image melanoma; however, each of these technologies may have inherent limitations and the optimal imaging parameters for the detection of melanoma have not been clearly established.

However, penetration depth reaching at least the papillary dermis is necessary to detect the melanoma invasion and differentiate invasive melanoma from melanoma in-situ. Resolution at the cellular level is desirable to make the diagnosis based on the histological differences between benign and malignant melanocytes, however lower resolution devices can still be used for detecting architectural differences between melanoma and benign nevi. Shortcomings of the various imaging systems may be as follows: Dermoscopy, depends on the appearance of classic dermoscopic features and therefore may have limited utility in the diagnosis of very early and mainly featureless melanomas. Dermoscopy also may not plan the excision since the margins of the excision rely on the Breslow depth. Multispectral imaging captures image data within specific wavelength ranges across the electromagnetic spectrum, this data however is projected on the same plane, obscuring depth information. Reflectance confocal microscopy provides cellular information on melanocytic lesions, however its penetration depth may be too limited to detect invasive melanoma. High-frequency ultrasound has generally a satisfactory penetration depth to detect the size and shape of a tumor, but the low resolution and low specificity may preclude diagnosis of the actual type of malignancy.

Raster scanning photoacoustic (PA) microscopy and cross-sectional PA tomography have been explored for diagnosis and staging of melanoma, in which melanin serves as an endogenous contrast agent. However, melanin is not a tumor specific biomarker of melanoma as it is present in benign nevi and may actually be absent in amelanotic melanoma. There have been several melanoma detection devices that may assist clinicians with any level of experience in the detection of melanoma, and subsequently rely on histopathological assessment.

Traditional devices, however, may have various drawbacks that can result in limited specificity and/or sensitivity thereby providing limited benefit to the clinician. Typical devices may utilize visible or near-infra-red (NIR) cameras. These longer wavelengths images provide sub-surface details, however, the results are reported from all layers simultaneously and thus obscuring essential depth information. Other devices may utilize Raman spectroscopy to analyze the chemical “fingerprint” of the lesion but this has no depth discrimination. Typical approaches are inadequate for melanoma.

Typical devices may also include a non-optical machine that analyses the electrical impedance spectrum of a lesion detected from tiny electrodes inserted into the tissue, which may not accurately differentiate nevi from melanoma. This has challenges in balancing sensitivity and specificity. Maximum sensitivity reduces the possibly missing a potentially fatal melanoma, but typically results in an unacceptably high false-positive rate from benign lesions due to poor specificity. This offers little benefit over traditional dermoscopy and clinician experience. Some of these devices can produce a “risk” measurement for diagnosing melanoma, but the user is required to subjectively decide the acceptable risk level. There has been a persistent unmet need for a melanoma diagnosis device with improved sensitivity and specificity.

Malignant melanoma is by far the most dangerous type of skin cancer. The initial step in a physician's decision to biopsy a suspicious lesion is dermoscopic inspection using the ABCDE criteria. A lesion that apparently fulfills the ABCDE criteria for melanoma is biopsied for definitive histopathologic diagnosis. Several non-invasive imaging approaches have been developed for the diagnosis of melanoma and differentiation from benign nevi. Their clinical utility, however, is limited because they do not provide sufficient specificity and sensitivity.

Standard techniques of diagnosing melanoma by excisional biopsy and histopathologic analysis requires approximately 15-30 benign lesions to be biopsied to diagnose each melanoma. Additionally, biopsies are invasive and result in pain, anxiety, scarring and disfigurement of patients, and can be a financial burden to the health care system.

Tissues have intrinsic scattering characteristics based on the density, size and shape of tissue microstructures, absorption characteristics derived from chromophore concentration, and anisotropy factor which correlates to cell size and disorder. These characteristics are modified during tumor development. Methods which can uniquely identify these characteristics hold promise for providing diagnostic value. Typical techniques lack the sensitivity and specificity in differentiating morphologically similar structures due to the interrelationships of these optical characteristics.

There is a need for improvements in melanoma detection as traditional systems and methods lack specificity and accuracy and are ultimately inadequate. The systems, devices and methods disclosed herein provide these improvements with solutions to the problems in traditional systems and methods.

### BRIEF DESCRIPTION OF THE DRAWINGS

The accompanying drawings are part of this specification and provide exemplary embodiments of this disclosure as follows:

FIGS. 1A, 1B, 1C and 1D illustrate an exemplary system of the present disclosure, for example, for identifying and detecting melanoma using optical coherence tomography (OCT);

FIG. 2 illustrates an exemplary process of the present disclosure including, for example, training and test phases;

FIG. 3 illustrates exemplary display screens of the present disclosure including, for example, mappings of optical information;

FIG. 4 illustrates exemplary display screens of the present disclosure including, for example, mappings of optical information including scattering coefficients, absorption coefficients, and anisotropy factors;

FIG. 5 illustrates an exemplary display screen of the present disclosure including, for example, mappings of optical information including sensitivity, specificity, Jaccard Index, and accuracy;

FIG. 6 illustrates an exemplary display screen of the present disclosure including, for example, mappings of optical information including subject index and melanoma, nevi and normal results;

FIG. 7 illustrates exemplary display screens of the present disclosure including, for example, mappings of optical information including imaging signal amplitude relative to melanoma and nevus results;

FIG. 8 illustrates an exemplary process of the present disclosure;

FIG. 9 illustrates an exemplary display screen of the present disclosure including, for example, mappings of optical information for multiple subjects;

FIG. 10 illustrates another exemplary display screen of the present disclosure including, for example, mappings of optical information for multiple subjects;

FIG. 11 illustrates exemplary user interface display screens of the present disclosure including, for example, mappings of optical information including scattering coefficients, absorption coefficients, and anisotropy factors;

FIG. 12 illustrates exemplary display screens of the present disclosure including, for example, mappings of optical information including scattering coefficients, absorption coefficients, and anisotropy factors;

FIG. 13 illustrates exemplary display screens of the present disclosure including, for example, mappings of optical information including sensitively, specificity, Jaccard, and accuracy data according to features permutated;

FIG. 14 illustrates an exemplary display screen of the present disclosure including, for example, mappings of optical information including false and true positive rates;

FIG. 15 illustrates an exemplary display screen of the present disclosure including, for example, mappings of optical information including the area under the curve (AUC) for each margin;

FIG. 16 illustrates an exemplary display screen of the present disclosure including, for example, mappings of optical information ink concentration percentage relative to absorption coefficient.

FIG. 17 illustrates exemplary display screens of the present disclosure including, for example, mappings of optical information including first, second and third methods and respective scattering coefficients, absorption coefficients, anisotropy factors, and error percentages;

FIG. 18 illustrates exemplary display screens of the present disclosure including, for example, mappings of optical information including imaging amplitude, scattering coefficient, absorption coefficient, and anisotropy factor; and

FIG. 19 includes exemplary display screens of the present disclosure including, for example, mappings of optical information including scattering coefficient, absorption coefficient, and anisotropy factor.

FIGS. 20A, 20B, and 20C include details of patients, lesion types, and locations, e.g., a list of melanoma and benign nevus cases for the methods herein.

### DETAILED DESCRIPTION

This disclosure provides improvements in optical coherence tomography (OCT) to overcome the shortcomings of traditional devices and techniques. This includes systems, devices and methods with advantages and solutions not provided by prior attempts. OCT systems may provide high spatial resolution (<10 microns), intermediate penetration depth (˜1.5 to 2 mm), and volumetric imaging capability. This disclosure provides a diagnostic-assistant modality in dermatology especially to detect and diagnose benign skin tumors, e.g., basal cell carcinoma (BCC) and squamous cell carcinoma (SCC). Interferometry may be used to record an optical path length of received photons, allowing rejection of most photons that scatter multiple times before detection. White light or a low coherence source may be split and recombined from a target tissue area and a healthy tissue area, e.g., target and healthy areas of an arm. The pathlength of the reference arm is varied in time, by moving or translating a reference mirror, and interference occurs when the pathlength difference lies within the coherence length of the light source. This disclosure provides adaptive systems and operations that leverage OCT imaging to gather patient-specific optical information and leverage aggregated results to optimize the accuracy and specificity of diagnostic results.

This disclosure provides improvements in OCT identification, detection and diagnosis of cancer such as melanoma. Contrast in OCT images is generated by the intrinsic scattering characteristics of tissue that are proportional to the density, size and shape of the tissue microstructures. Because malignant cells show pleomorphism, with different refractive indices and absorption characteristics than normal cells, based on light-tissue interaction theories, typical OCT techniques do not have the specificity to discriminate malignant tissues from normal tissues and benign neoplasms. To overcome the shortcomings of prior attempts, this disclosure provides improved specificity, image enhancement and texture analysis as well as sophisticated configurations of OCT. The present disclosure provides advantages over existing polarization-sensitive, phase-sensitive and dynamic OCT systems by providing improved imaging that accurately discriminates between melanoma and benign lesions. This disclosure also includes improvements that overcome the aggregation of the predominant optical properties that contribute to OCT image formation and thus preserves and improves the specificity of melanoma detection.

The systems, devices and methods herein include imaging techniques to enhance melanoma diagnosis and OCT imaging. The systems herein may utilize OCT with high-resolution and intermediate penetration depth provide improved diagnostic information, e.g., noninvasively. The methods may include an image analysis method including optical properties extraction (OPE). This may improve the specificity and sensitivity of OCT and diagnostic accuracy, e.g., by identifying unique optical radiomic signatures pertinent to melanoma detection and differentiating melanoma from benign nevi. The present disclosure provides improvements to, among other things, the sensitivity and specificity of the detection and diagnosis of melanoma.

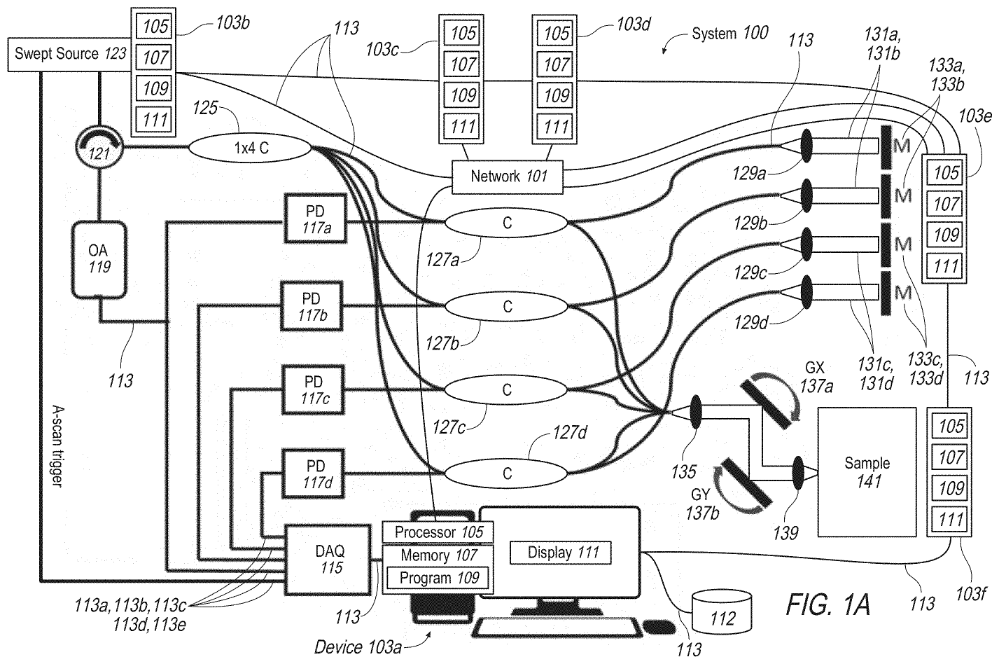

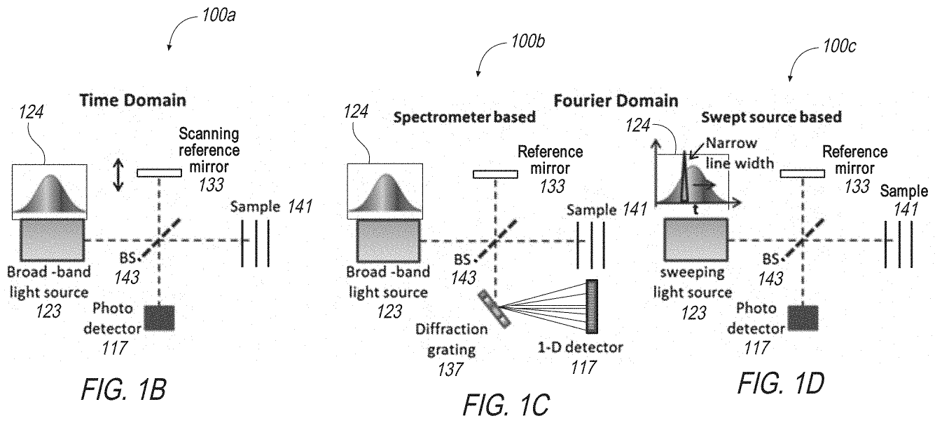

Referring to FIGS. 1A, 1B, 1C and 1D, system 100 may include an imaging system configured to provide the operations herein, e.g., for identifying, distinguishing and diagnosing optical information of patient-specific tissue areas. System 100, for example, may be configured as a multi-beam, swept-source OCT (SS-OCT) system including computing device 103 communicatively connected with swept source 124, e.g., a scanning probe such as a hand-held scanning probe for skin imaging.

With reference again to FIG. 1A, system 100 may include one or a plurality of components such as network 101, device 103 (e.g., computing devices 103a,b,c,d,e,f), processor 105, memory 107, program 109, display 111, database 112, connection 113 (e.g., 113a,b,cd,e), data acquisition (DAQ) device 115, detector 117 (e.g., photodetector (PD) 117a,b,c,d), optical attenuator (OA) 119, dial 121 (e.g., rheostat, variable resistor, or potentiometer), source device 123 (e.g., swept-source OCT laser, broad-band light source, or sweeping light source), device 125, optical coupler 127 (e.g., 127a,b,c,d), lens 129 (e.g., 129a,b,c,d), device 131 (e.g., 131a,b,c,d), fixed, scanning or adjustable mirrors (M) 133 (e.g., 133a,b,c,d), lens 135, galvanometer 137 (e.g., x-axis galvanometer 137a, y-axis galvanometer 137b or diffraction grating), lens 139, and sampling device 141. Any or all of the components of system 100 may receive, retrieve, store, generate, aggregate, disaggregate, display, send, communicate, and/or transfer data (e.g., optical information) with respect to any other component of system 100, e.g., by way of any or all of network 101, devices 103, processor 105, memory 107, program 109, display 111, database 113, and connections 113, to provide any or all of the operations and data (e.g., optical information) disclosed herein.

With reference to FIGS. 1B-D, system 100 may include system 100 may generate one or a plurality of scans (e.g., first and second images.). System 100 may generate and/or receive scans including A-scans, B-scans, C-scans, time domain scans, Fourier-domain (FD) scans (e.g., spectrometer or swept source based), spectral-domain (SD) scans, three-dimensional (3D) scans, or a combination thereof. System 100 may include system 100a configured for time domain scans, system 100b configured for spectrometer-based scans (e.g., Fourier domain), system 100c configured for swept source-based scans (e.g., Fourier domain), or a combination thereof. System 100 may include a broad-band (FIGS. 1B-C) or sweeping light source (FIG. 1D), a photo detector (FIGS. 1B and D) or diffraction grating in optical communication with a detector (e.g., one dimensional) (FIG. 1C), a scanning reference mirror at an adjustable distance (FIG. 1B) or a fixed distance (FIG. 1C), a beam splitter 143, or a combination thereof.

In embodiments, systems, devices and methods may use optical coherence tomography (OCT) to identify a skin lesion. System 100, and corresponding devices and methods, may include scanning probe 123 configured to image skin and computing device 103 having a hardware processor 105 and physical memory 107. Scanning probe 123 and computing device 103 maybe communicatively connected to each other to provide operation. The operations may include, for example, obtain a first image of a suspect region-of-interest (SROI) for a suspect lesion, obtain a second image of a healthy region-of-interest (HROI) near the suspect lesion, classify the extracted optical properties to generate an issue status including as at least one of melanoma tissue and benign tissue, and display the issue status indicating the at least one of melanoma tissue and benign tissue. The operations may further include to normalize optical properties from the SROI and from the HROI, obtain an averaged A-line of the SROI and the HROI, generate a set of normalized optical radiomic features from an averaged A-line of the SROI and the HROI, evaluate the set of normalized optical radiomic features to distinguish whether the suspect lesion is consistent with the melanoma tissue and the benign tissue, and display optical information including at least one of optical properties, normalized optical properties, and classified optical properties indicating the least one of melanoma tissue and benign tissue. The first and second images may include at least one of A-scans, B-scans, C-scans, Fourier-domain (FD) scans, spectral-domain (SD) scans, and three-dimensional (3D) scans.

Embodiments may include systems, devices and methods for identifying a skin lesion. System 100, and corresponding devices and methods, may include scanning probe 123 for skin imaging and computing device 103 to provide the operations herein. The operations may include to define a suspect region-of-interest (SROI) for a suspect lesion in a first OCT B-scan image, define a healthy region-of-interest (HROI) near the suspect lesion in a second OCT B-scan image, extract optical properties from the SROI and from the HROI, obtain an averaged A-line in the SROI and in the HROI, create a set of normalized optical radiomic features from the averaged A-line in the SROI and in the HROI, and evaluate the set of normalized optical radiomic features to distinguish whether the suspect lesion is consistent with melanoma.

Embodiments of system 100 may, for example, execute by processor 105 instructions of program 109 to provide optical information displayed on display 111. These instructions and operations may be retrieved from or stored on swept source 123, device 103, memory 107, database 112 or a combination thereof. Optical information may include an optical property extraction (OPE) method and apparatus, e.g., based on an Extended Huygens-Fresnel (EHF) model. System 100 may disaggregate by processor 105 an OCT image into its individual optical attributes, e.g., tissue scattering coefficient, absorption coefficient and anisotropy factor. System 100 may identify by processor 105 optical information such as unique optical radiomic signatures that are pertinent to melanoma detection among the extracted optical properties and trained heuristics (e.g., a machine-learning kernel). System 100 may utilize by processor 105 a detection method such as an optical radiomic melanoma detection (ORMD). System 100 may execute by processor 105 the detection method on OCT images of the suspect lesion to determine and display by display 111 optical information including diagnosis results, e.g., a tissue status. The tissue status may include non-melanoma, benign, or healthy tissue (e.g., “Tissue sample is consistent with healthy tissue”), or melanoma, malignant, or unhealthy tissue (e.g., “Tissue sample exhibits characteristics consistent with melanoma”).

Exemplary advantages of system 100 may include reducing the number of unnecessary biopsies. System 100 may identify the most probable malignant lesion in a person with one or multiple abnormal areas, e.g., pigmented spots. The advantages of system 100 include fewer biopsies and less pain, anxiety, scarring and disfigurement for patients. System 100 may be configured to detect melanoma in its early stage, e.g., while prognosis is optimal. System 100 may extract optical properties embedded in existing or real-time image data. System 100 may readily extract this before, during or after image processing.

System 100 may include swept source 123. System 100 may provide lateral and axial resolutions of about 7.5 μm and 10 μm, respectively. The scan area of system 100 may be 6 mm (width)×6 mm (length)×2 mm (depth), with a frame rate of 20 frames per second. System 100 may include a tunable broadband laser source with the central wavelength of 1305±15 nm successively sweeps through the optical spectrum and leads the light to four separate interferometers (e.g., four) and forms consecutive confocal gates (e.g., four).

System 100 may include an OPE method and/or EHF model. System 100 may utilize a light-tissue interaction specific to OCT imaging. OCT modeling may be initiated by considering a scattering coefficient for modeling using a single-scattering theory involving a ballistic component. The scattering coefficient and/or ballistic component may be used alone, with each other, or in combination with other optical or diagnostic information herein. System 100 may utilize single-scattering model and quantitative analysis of OCT images for potentially reduced signal decay with depth to provide improved diagnostic accuracy, or multiple scattering with potentially increased signal decay with depth.

System 100 may provide, by processor 105 executing instructions of program 109 stored on memory 107 and displayed on display 111, operations for optical information. This may include receiving inputs, generating outputs, and displaying diagnostic results based on such inputs and outputs. System 100 may utilize inputs such as a ballistic light component and multiple scattered light. System 100 may provide an analytical solution to the scalar wave equation based on mutual coherence functions using, e.g., the Extended Huygens-Fresnel (EHF) principle. This may include diffraction effects and/or allow a Gaussian beam under any focusing condition. A lateral coherence length variation has been integrated with depth into previous models by considering a “shower curtain effect.” System 100 may describe the heterodyne OCT signal as a function of depth and incorporates both multiple scattering and single scattering effects. System 100 may utilize the EHF principle employed in an OCT model and in a multilayer-scattering geometry. Embodiments may include optical information such as the addition of a third parameter, absorption coefficient, scattering coefficient and anisotropy factor.

System 100 may generate by processor 105 optical information including a mean squared of the OCT heterodyne signal current at the probing depth z as follows (“equation 1”):

[symbol]

i²(z) [symbol] = [symbol] i² [symbol] ₀ψ_{SA}(z) (1)

where, [symbol] i² [symbol] =a/w_{H}² is the mean squared heterodyne signal current in the absence of scattering and absorption, a is a constant characterized by the OCT system setup and w_{H}² is 1/e irradiance radius at the probing depth in the absence of scattering as follows (“equation 2”):

$${w}_{H}^{2} = {{w}_{0}^{2} ( A - \frac{B}{f} )}^{2} + {( \frac{B}{{kw}_{0}} )}^{2} \qquad ( 2 )$$

where, A and B are the elements of ABCD matrix for light propagation from the lens plane to the probing depth in the sample. If the focal plane of the beam is fixed on the surface of the sample, then A=1 and B=f+z/n, where n is the refractive index, and f is the focal length of the lens, w₀ represents the 1/e irradiance radius of the input sample beam at the lens plane. k=2π/λ, and λ is the wavelength of light source. ψ_{SA}(z) is the heterodyne efficiency factor describing signal degradation due to scattering and absorption as follows (“equation 3”):

$${ψ}_{SA} ( z ) = {e}^{- 2 {μ}_{a} z} [ {e}^{- 2 {μ}_{s} z} + \frac{4 {e}^{- {μ}_{s} z} [ 1 - {e}^{- {μ}_{s} z} ]}{( 1 + {μ}_{a} Δ {z}_{D} ) ( 1 + ( \frac{{w}_{SA}^{2}}{{w}_{H}^{2}} ) )} + \frac{{( 1 - {e}^{- {u}_{s} z} )}^{2} {w}_{H}^{2}}{{( 1 + {μ}_{a} Δ {z}_{D} )}^{2} {w}_{SA}^{2}} ] \qquad ( 3 )$$

The first term in the brackets represents the single scattering effect, the third term is the multiple-scattering term, and the second term is the cross term including both single and multiple scattering. w_{sA} is the 1/e irradiance radius at the probing depth in the presence of scattering and absorption as follows (“equation 4”):

$${W}_{SA}^{2} = {( 1 + {μ}_{a} Δ {z}_{D} )}^{- 1} [ {{w}_{0}^{2} ( A - \frac{B}{f} )}^{2} + \frac{{B}^{2}}{{kw}_{0}} + {( \frac{2 B}{k {ρ}_{0}} )}^{2} ( i + {μ}_{A} Δ {z}_{N} ) ] \qquad ( 4 )$$

where ρ₀ is the lateral coherence length as follows (“equation 5”):

$${ρ}_{0} = \sqrt{\frac{3}{{μ}_{s} z}} \frac{λ}{{πθ}_{rms}} ( 1 + \frac{{n}_{R} d ( z )}{z} ) \qquad ( 5 )$$

where θᵣₘₛ is the root mean squared scattering angle, defined as the half-width at 1/e maximum of a Gaussian curve fitted to the main frontal lobe of the scattering phase function, and n_{R} is the real part of refractive index. Also, Δz_{N}, and Δz_{D} are as follows (“equation 6” and “equation 7,” respectively):

$$\begin{matrix}Δ {z}_{N} = \frac{z ( {w}_{0}^{2} + \frac{{ρ}_{0}^{2}}{2} )}{4 {n}_{R}^{2} {B}^{2}} & ( 6 ) \\ Δ {z}_{D} = \frac{z}{2 {n}_{R}^{2}} [ {( \frac{{w}_{0}}{f} )}^{2} + {( \frac{1}{{kw}_{0}} )}^{2} + {( \frac{2}{k {ρ}_{0}} )}^{2} ] & ( 7 )\end{matrix}$$

Any or all of equations herein may be executed by processor 105 using any inputs or providing any outputs herein. This may include displaying optical information by display 111, e.g., the inputs being w₀, λ, and f and/or the outputs being optical properties such as a scattering coefficient, an absorption coefficients, and an anisotropy factor.

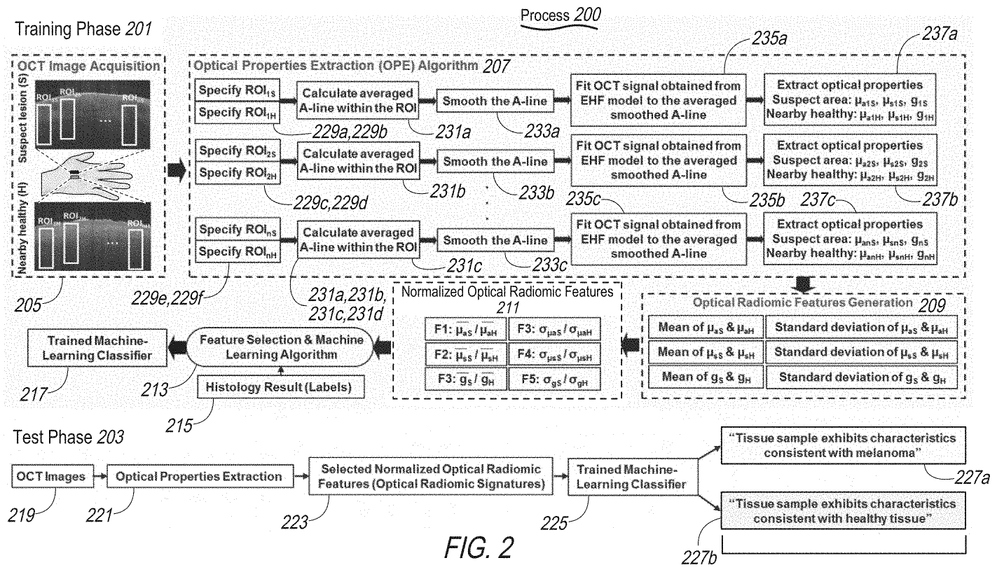

FIG. 2 illustrates process 200 that may be executed by processor 105, stored on memory 107 or database 112, and/or displayed on display 111. Process 200 may include an optical radiomic melanoma detection (ORMD), optical properties extraction (OPE), or a combination thereof. Process 200 may include inputs such as a scattering coefficient (μₛ); absorption coefficient (μₐ), and anisotropy factor (g). Process 200 may include selecting a region of interest (ROI) in a preprocessed B-scan OCT image with the pre-processing details set forth below. For example, a selection area (e.g., shown as rectangles) may demarcate a selected ROI from which the optical properties are calculated. The pixel intensities along the x-axis in the ROI are averaged to obtain an averaged A-line. For the fitting, process 200 may execute a modified and/or exhaustive search of the optical information.

Process 200, by way of memory 105, database 112, device 103, and/or swept source 123, may include receiving or obtaining OCT images from suspect lesion 205a and nearby healthy skin region 205b. Process 200 may include specifying a region of interest (ROI) of an OCT image, e.g., on an OCT B-scan image. Process 200 may average pixel intensities along the x-axis in each ROI obtain or provide an averaged A-line. Process 200 may include fitting the scattering and absorption coefficients and the anisotropy factor in a modeled OCT signal, adjusting these in the modeled OCT signal, and providing or displaying by display 111 a curve that best fits the averaged A-line.

Process 200 may perform one or multiple iterations by repeating this for several regions of interest (ROIs), average the iterations, and generate standard deviations for the interactions. Process 200 may derive optical information such as radiomic features for that tissue including, for example, mean and standard deviation of scattering and absorption coefficients, and anisotropy factor. The optical information including these radiomic features obtained by system 100 from the suspect lesion and its nearby healthy skin are used to generate a set of normalized optical radiomic features based on gender, age and skin color.

The system 100 may provide, by processor 105 executing instructions of program 109 on memory 107 or database 112, classification operations for optical information. This may include heuristics (e.g., machine learning) as part of a supervised, unsupervised or automated classification between optical radiomic features of cancerous or melanoma tissue and non-melanoma, benign, or healthy tissue to provide improved long-term results for the widest variation of melanoma types and stages. System 100 may histologically compare and validate optical information, e.g., inputs, outputs and/or results. System 100 may adapt operations, manually with or automatically without human intervention, to detect nuanced variations in cytology to identify melanoma from its first detectable inception. The system 100 may include a priori knowledge of OCT images and, healthy and melanoma tissue histology, to allow training of heuristics (e.g., a machine-learning kernel) with improved specificity and detection accuracy than traditional systems and classifiers.

Referring again to FIG. 2, process 200 may include one or multiple phases such as Training Phase 201 and Test Phase 203. For Training Phase 201, the optical radiomic features and their labels (e.g., histology results) are input to provide heuristics, e.g., machine learning. For Test Phase 203, OCT images of a suspect skin area will be analyzed by the trained heuristics (e.g., machine-learning kernel) with the selected optical radiomic features, e.g., optical radiomic signatures, and indicate or display diagnostic results using display 111. Diagnostic results may include the tissue status such as healthy, non-cancerous, non-melanoma, or benign tissue (e.g., “Tissue sample is consistent with healthy tissue”), or unhealthy, cancerous, melanoma or malignant tissue (e.g., “Tissue sample exhibits characteristics consistent with melanoma”).

For training phase 201 at block 205, process 200 may include obtaining optical information including a first OCT image of a suspect area (e.g., legion) and a second OCT image of a nearby healthy area (e.g., normal tissue). At block 207, process 200 may include and generate optical properties extraction (OPE). At block 209, process 200 may generate optical radiomic features from each pair of suspect and health areas. At block 211, process 200 may normalize optical radiomic features. At blocks 213 and 215, process 200 may include feature selection and heuristics (e.g., machine learning) based on histology results (e.g., labels). At block 217, process 200 may include generating trained heuristics (e.g., machine-learning) classifiers. After block 217, process 200 may restart training phase 201 at block 205, proceed with test phase 203 at block 219, or it may end.

For test phase 203 at block 219, process 200 may include receiving OCT images. At block 221, process 200 may extract optical properties. At block 223, process 200 may generate a comparison with selected normalized optical radiomic features (e.g., optical radiometric signatures). At block 225, process 200 may generate a comparison with trained heuristics (e.g., machine-learning) classifiers. At block 227, process 200 may indicate or display on display 111 the tissue status indicating melanoma at block 227a or healthy at block 227b. After blocks 227, process 200 may restart test phase 203 at block 227, restart training phase 201 at block 205, or it may end.

Block 207 may include optical properties extraction (OPE) with a plurality of operations. At blocks 229a,b,c,d,e,f, process 200 may include specify ROIs. At blocks 231a,b,c, process 200 may calculate averaged A-line within each ROI. At blocks 233a,b,c, process 200 may include smooth the A-line. At blocks 235a,b,c, process 200 may fit the OCT signal obtained from the EHF model to the averaged and smoothed A-line. At blocks 237a,b,c, process 200 may extract optical properties of suspect area and nearby healthy area.

System 100, by way of processor 105, may generate a fitting error using l₁ norm as follows (“equation 8”):

$$Error = \frac{100}{n} {\sum }_{i = 1}^{n} \vert \frac{{signal}_{OCT} ( i ) - {signal}_{model} ( i )}{{signal}_{OCT} ( i )} \vert \qquad ( 8 )$$

n is the number of signal elements, i is the pixel index in depth, signal_{OCT}(i) is the averaged OCT A-line, signal_{model}(i) is the corresponding EHF model heterodyne signal, which may be calculated from equation 1 above. A smaller error correlates to a better fit and more robust results.

The system 100 may be configured for statistical and/or adaptive analysis of optical information. System 100 may test the global difference among the experimental settings. The null hypothesis may be that there is no difference among the experiment settings. For similarity measure, an equivalence test at 5% level of significance is used, in this example. The null hypothesis may be that the absolute difference between the means of two experimental settings is larger or equal to a threshold value, A. (e.g., H₀:|mean_{A}−mean_{B}|≥Δ). Different values of delta may be chosen for different settings and the values may be based on preliminary results for clinical importance. The rejection of the null hypothesis indicates the equivalence of the two conditions. The other statistical tests may be two sided at the 5% level of significance.

System 100 may include phantom operations for optical information. To evaluate the OPE method, phantoms may be created using first and second materials (e.g., milk and ink) with optical characteristics similar to skin. The advantages of milk are its predetermined optical properties, the similarity of its micro particles to organelles that constitute the scattering sources in tissue, and its homogeneity and accessibility at different concentrations. Various concentrations of milk (e.g., organic milk) may be obtained by mixing it with varying quantities of distilled water and India ink to make milk and milk-ink phantoms.

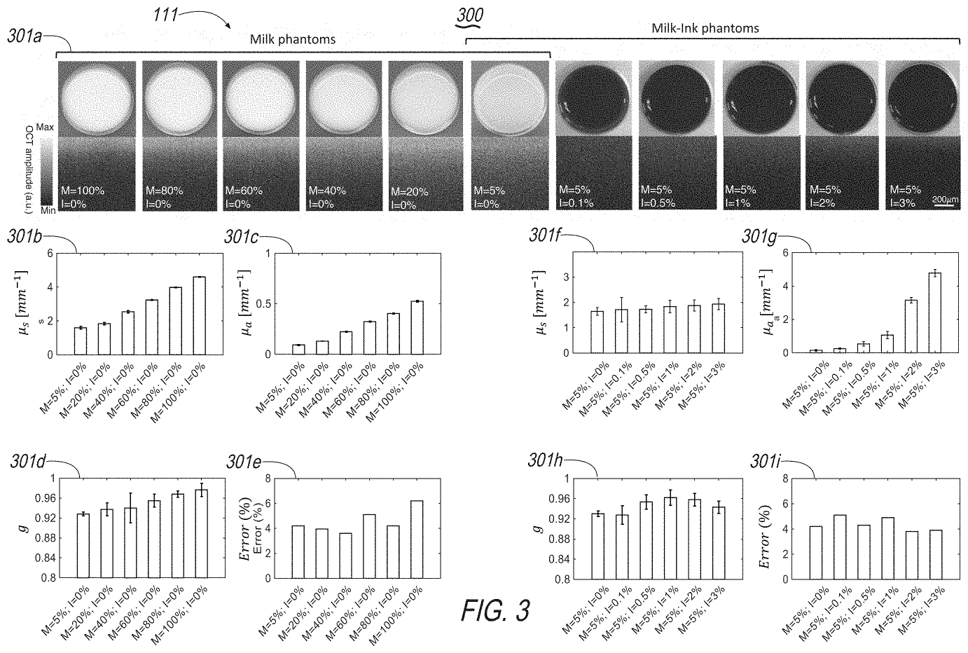

Referring to FIG. 3, process 300 of system 100 may include a phantom process with optical information, e.g., generated by processor 105 and displayed on display 111. Process 300 may include optical information 301a including photographic and OCT images of milk and milk-ink phantoms, optical information 301b,f may include scattering coefficients (μₛ), optical information 301c,g may include displaying absorption coefficients (μₐ), optical information 301d,h may include anisotropy factors (g), optical information 301e,i may include fitting error. This may include indicator one (e.g., *) with p<0.001 and indicator two (e.g., **) with p<0.01. Each x-axis shows the concentration of milk diluted by water with M and I showing the concentration of milk and ink diluted by water.

Process 300 may include inputs such as concentrations of milk in water including 5%, 20%, 40%, 60%, 80% and 100%, and those of ink may be 0%, 0.1%, 0.5%, 1%, 2%, and 3%. Percentages of milk, ink and water in milk and milk-ink phantoms, e.g., Table 1 below corresponding to the phantoms in FIG. 3 from left to right.

TABLE 1

| | | | | | | | | | | | |
|---|---|---|---|---|---|---|---|---|---|---|---|
| Milk (%) | 100 | 80 | 60 | 40 | 20 | 5 | 5 | 5 | 5 | 5 | 5 |
| Ink (%) | 0 | 0 | 0 | 0 | 0 | 0 | 0.1 | 0.5 | 1 | 2 | 3 |
| Distilled | 0 | 20 | 40 | 60 | 80 | 95 | 94.9 | 94.5 | 94 | 93 | 92 |
| Water (%) | | | | | | | | | | | |

The photographic and OCT images of the phantoms and the values of the scattering coefficients, absorption coefficients, anisotropy factors, and error bars for 10 runs. All data and tables herein are provided as exemplary embodiments, and other data and data ranges are contemplated.

System 100 may include in-vivo operations. System 100 may include a motorized, triaxial holder to secure swept source 123 (e.g., an OCT probe) and ensure stability during imaging. Swept source 123 may be placed in the middle of the suspected lesion, based on the bright-field image provided by miniaturized camera integral to the OCT system and the red indicator beam. Swept source 123 may include a predefined or user-defined volume such as 6 mm (L)×6 mm (W)×2 mm (D) may be scanned and 600 cross-section images with 10 μm span may be generated.

System 100 may utilize inclusion and exclusion criteria. For example, inclusion criteria may include (1) age 18 years or older; (2) able to provide written informed consent prior to any trial-related procedure. Exclusion criteria may include, for example, (1) failure to give informed consent; (2) anatomic site of the lesion not accessible to the device; (3) lesion previously biopsied, excised, or traumatized; (4) skin not intact (e.g., open sores, ulcers, bleeding); (5) lesion on palmar, plantar, or mucosal (e.g., lips, genitals) surface or under nails; (6) lesion containing foreign matter (e.g., tattoo ink, splinter, marker).

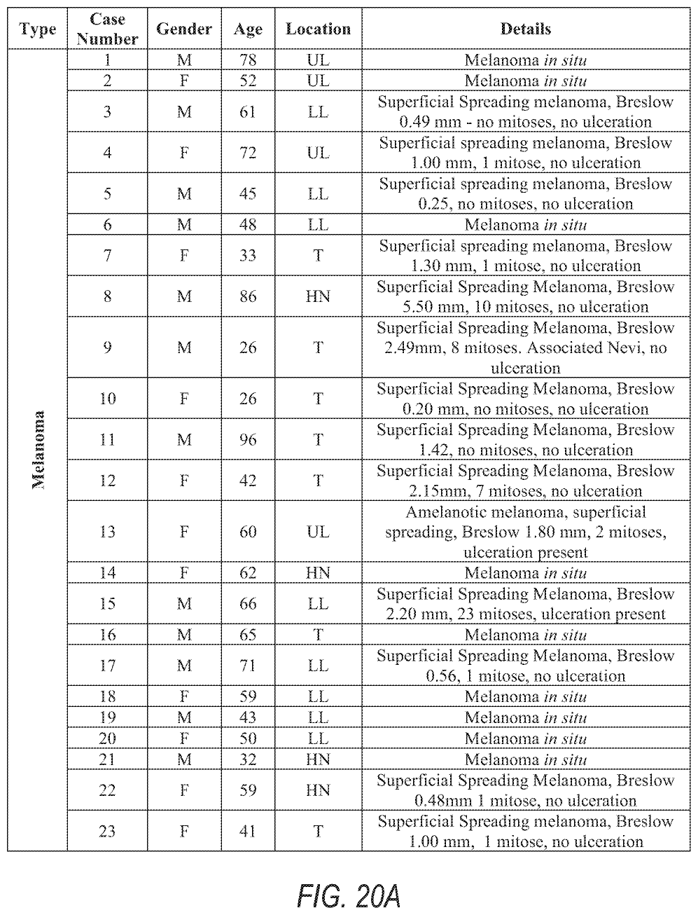

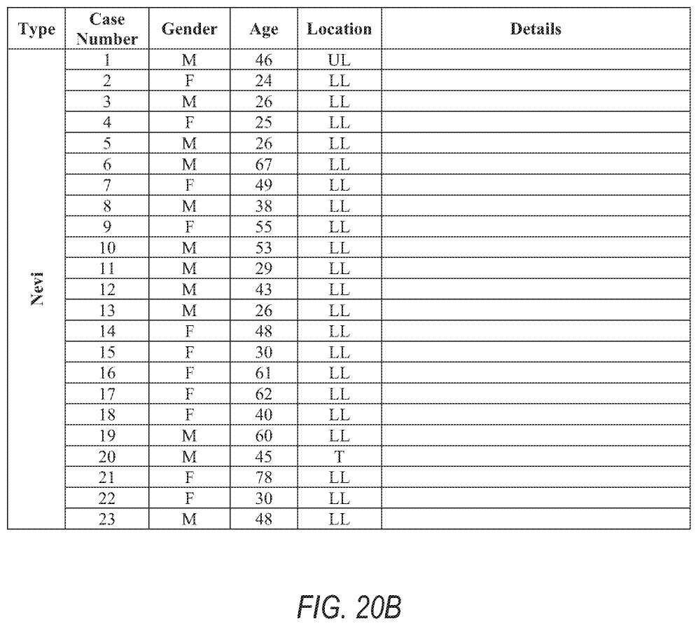

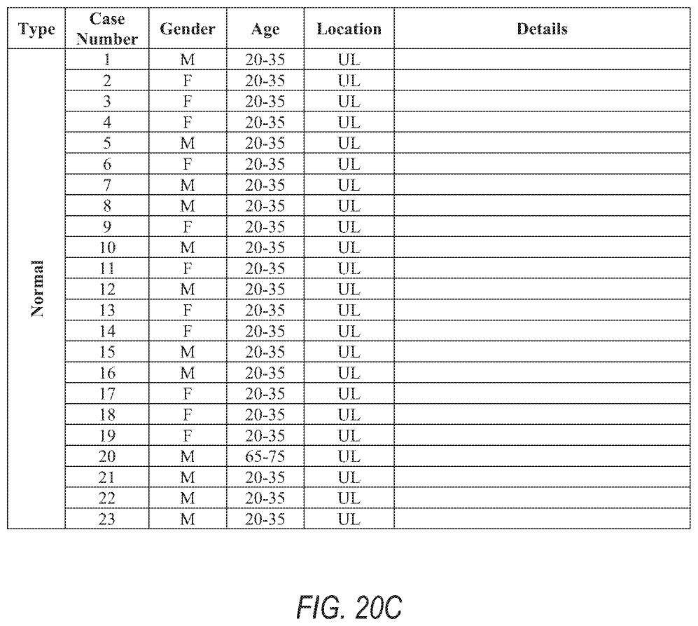

System 100 may receive, by way of swept source 123, device 103, memory 107 or database 112, OCT images from a predefined number of subjects, e.g., aged between 20 to 80 years and/or from a high-risk dermatology clinic. This may include a number of samples biopsied from respective patients having healthy skin, a variety of benign nevi and at least one suspect melanoma lesion. The tables of FIGS. 20A, 20B, and 20C include details of patients, lesion types and locations, e.g., a list of melanoma and benign nevus cases for the methods herein female (F), male (M), upper limbs (UL), lower limbs (LL), head and neck (HN), and trunk (T).

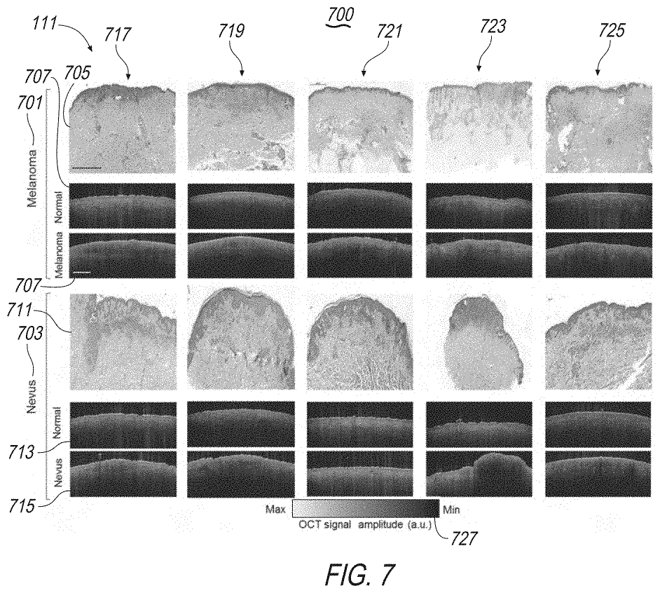

As shown in FIG. 7, system 100 may image, by swept source 123, each of the melanoma or benign nevi and adjacent healthy skin a control. System 100 may analyze and compare the cases and reported the histopathological findings in accordance with a predefined standard of care. System 100 may store, on memory 107 or database 112, the histology image of the suspected area and OCT images of healthy and diseased regions. OCT images and histology photographs for ten selected melanoma and benign nevi cases are shown in FIG. 7 together with OCT images of their nearby healthy skin.

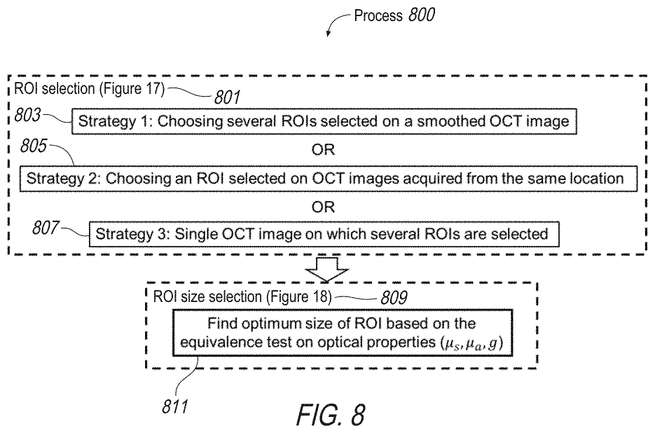

Referring to FIG. 8, system 100 execute process 800 by processor 105, referred to as “preprocessing.” The optical properties of healthy skin, melanoma, and benign nevi may be then extracted from the images as discussed herein. In the processing procedure, for each patient, three adjacent OCT images (e.g., 10 microns apart) from the melanoma/benign and three adjacent OCT images from their nearby healthy skin may be used for analysis. For each of these three images, a predefined number (e.g., 24) of ROIs may be specified, and optical properties of these ROIs may be calculated, e.g., the scattering coefficient, absorption coefficient and anisotropy factor. The mean and standard deviation of optical properties obtained from the three sets (e.g., 72) of ROIs of suspicious images may be reported as the optical properties of the imaged lesion and the three sets (e.g., 72) of ROIs of nearby healthy images may be reported as the optical properties of the imaged nearby healthy tissue.

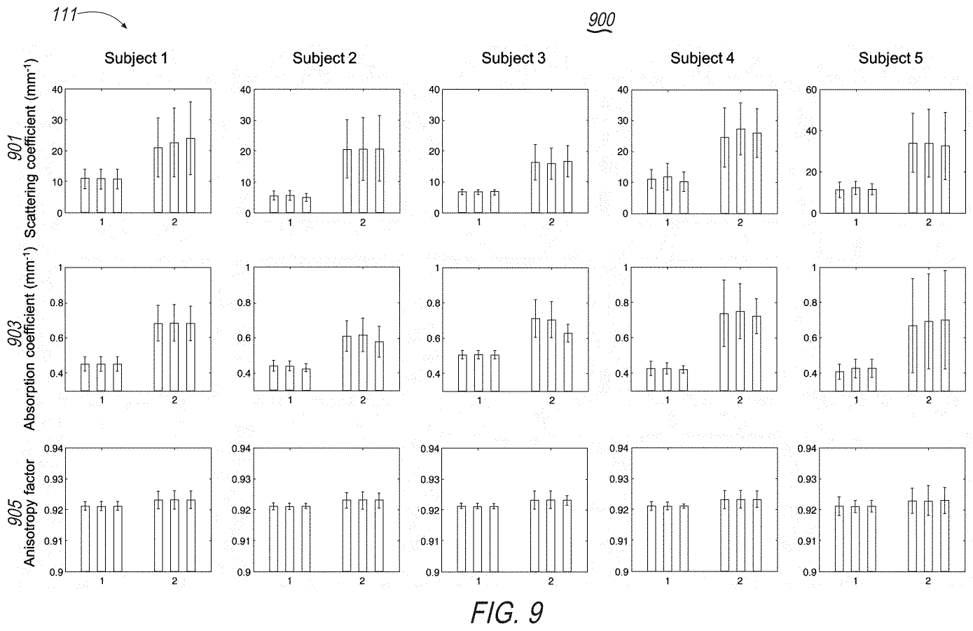

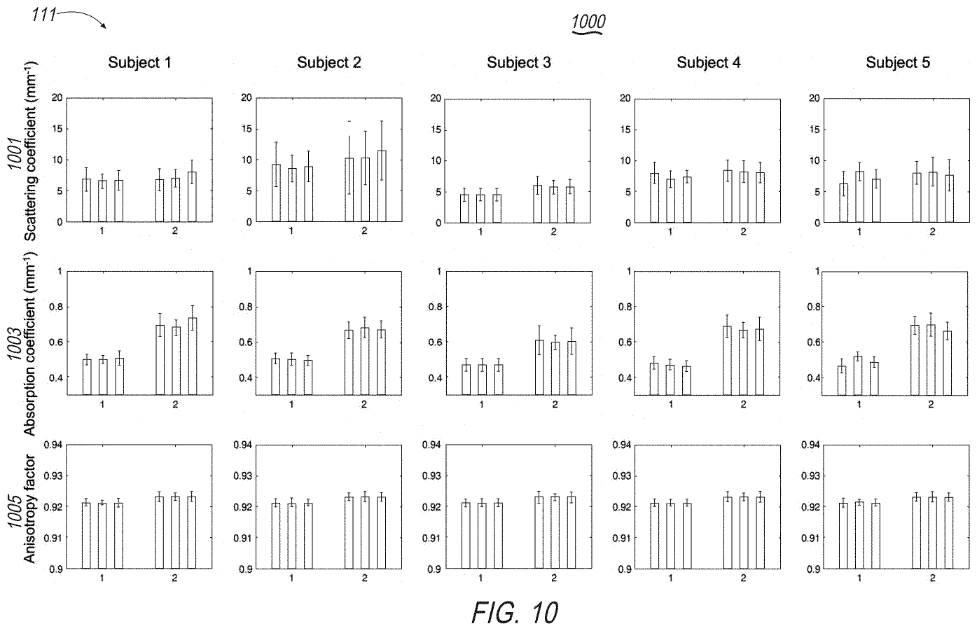

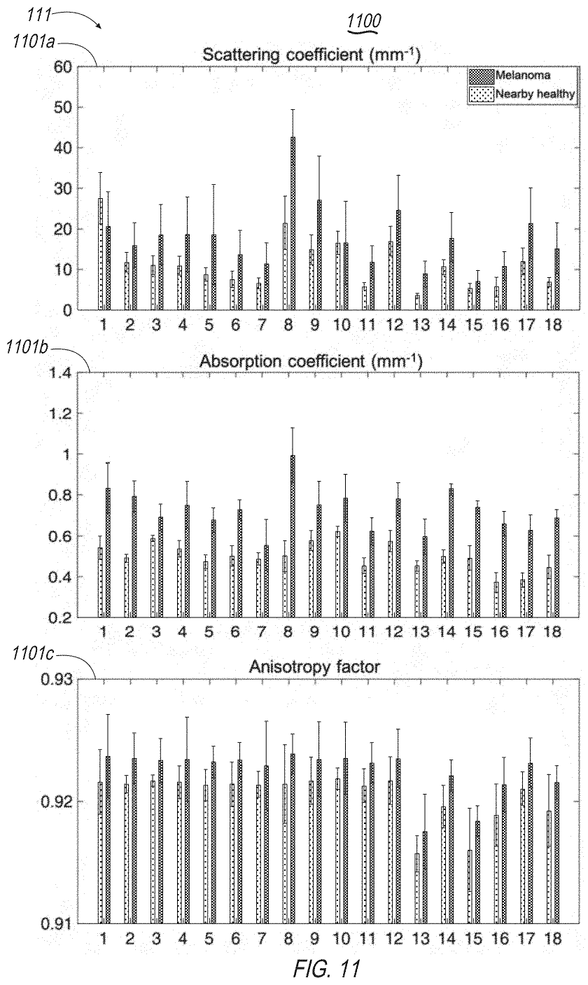

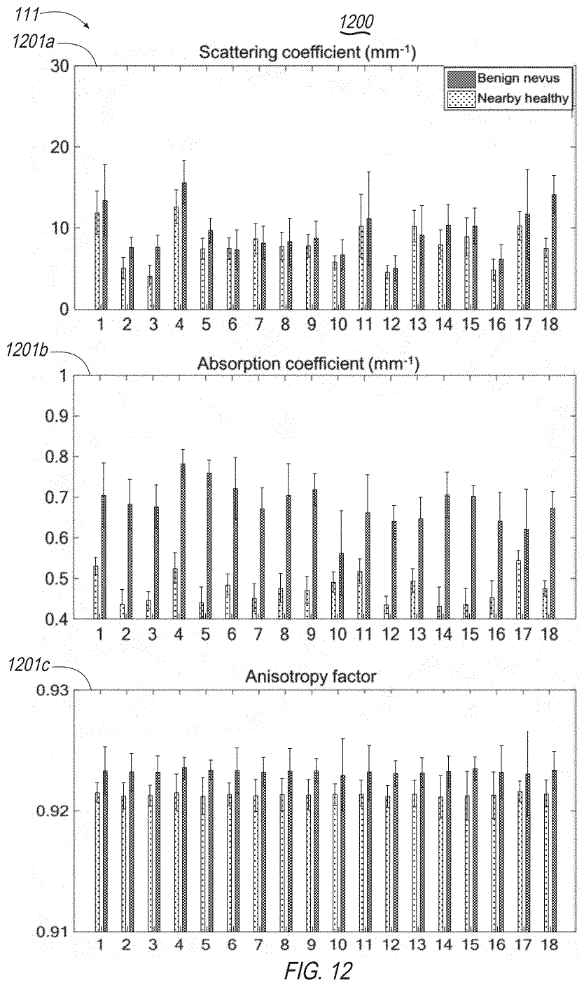

Referring again to FIG. 3, system 100 may generate, store and display outputs 300 including optical information 301a-i. This may include OPE-derived optical properties for a predefined (e.g., ten), arbitrarily selected cases of melanoma and benign nevi (e.g., five each), as well as their nearby healthy skin comparators, e.g., optical information 301a-f. Optical information 300g-i may include mean and standard deviation for the same patients to demonstrate, in general, how melanoma, and benign nevi skin differ for each optical property extracted. See also FIGS. 9 and 10. Optical information may also include extracted optical properties from the other cases (e.g., 36) of melanoma and nevi. See FIGS. 11 and 12.

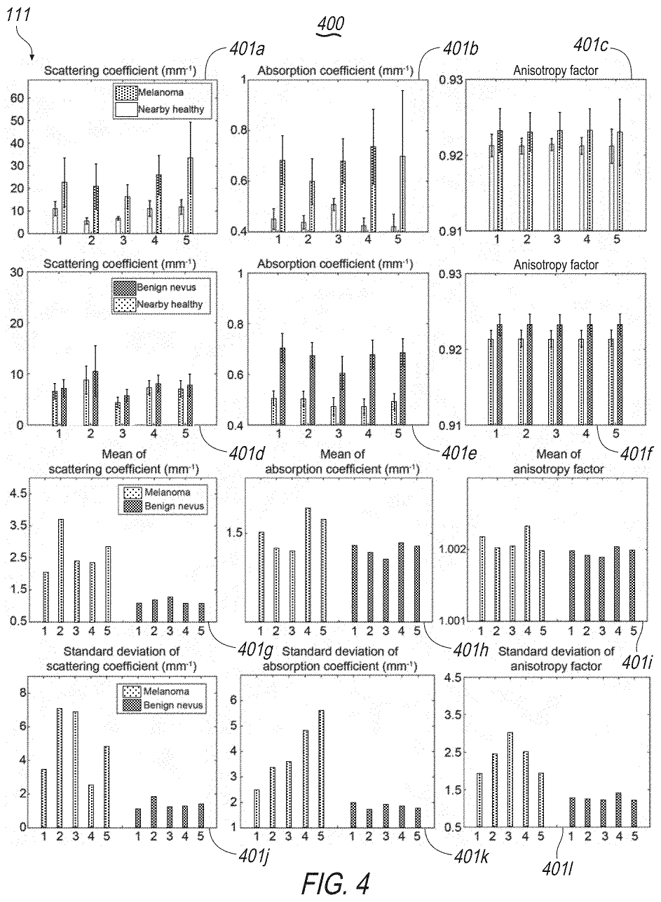

With reference to FIG. 4, system 100 may extract, generate, store and display outputs 400 including optical information 401. Optical information may include optical properties from OCT images of melanoma and benign nevi, and nearby healthy skin for a predefined (e.g., ten) arbitrarily selected subjects. Optical information may include any or all of scattering coefficients 401a, absorption coefficients 401b, anisotropy factor 401c of melanoma lesions and their nearby healthy skin, scattering coefficients 401d, absorption coefficients 401e, anisotropy factor 401f of benign nevi and their nearby healthy skin, side by side comparison 401g-1 of the optical properties normalized to nearby healthy tissue of melanoma versus benign nevi, normalized means 401g-i of scattering coefficient, absorption coefficient, and anisotropy factor 401i, respectively, and normalized standard deviations 401j-1 of scattering coefficient, absorption coefficient, and anisotropy factor, respectively.

System 100 include classification operations for optical information. For subjects with dermatologically identified benign nevi and malignant lesions, stacks of OCT images (e.g., 60), with a span of 10 μm, may be taken. Additionally, another stack of images may be taken of nearby healthy skin, at a minimum distance of 1.5 cm from the lesion, for data normalization and to compensate for factors related to skin type, age, and gender. The dorsal surface of the hand may be imaged for healthy subjects. From each stack, three images acquired from the center of the lesion may be selected and used for image analysis using the disclosed OPE method. For each lesion, six optical radiomic features may be obtained; F₁, F₂, F₃, the means of scattering coefficient, absorption coefficient, and anisotropy factor; and F₄, F₅, F₆, the standard deviations.

System 100 may provide operations using linear and non-linear classifiers including Linear Discriminant Analysis (LDA), Linear Regression (LR), K-Nearest Neighbor (KNN) with different K-values (K=1, 3, 5, and 7), Linear Support Vector Machine (LSVM), Quadratic SVM (QSVM) and Gaussian SVM (GSVM) for testing and identification possible combinations of features. For a smaller number of subjects, system 100 may utilize an n-fold cross validation method including folds (e.g., 20). Each classifier may be trained with random combinations (e.g., 20) of training and test datasets (e.g., 70% and 30%, respectively). The reported values are the average of 20 measurements, with mean and standard deviations.

System 100 may generate permutations of the previously obtained features. System 100 may combine these with each classifier, and its various configurations, numerous unique discriminators determine the best values for sensitivity. For example, system 100 may utilize Jaccard index and accuracy as set forth in Table 2 below, e.g., diagnostic statistics including sensitivity, specificity, Jaccard index and accuracy within indications including true positive (TP), true negative (TN), false positive (FP) and false negative (FN).

TABLE 2

| Statistic | Formula |
|---|---|
| Sensitivity | $\frac{TP}{TP + FN}$ |
| Specificity | $\frac{TN}{FP + TN}$ |
| Jaccard index | $\frac{TP}{TP + FN + FP}$ |
| Accuracy | $\frac{TP + TN}{TP + FP + TN + FN}$ |

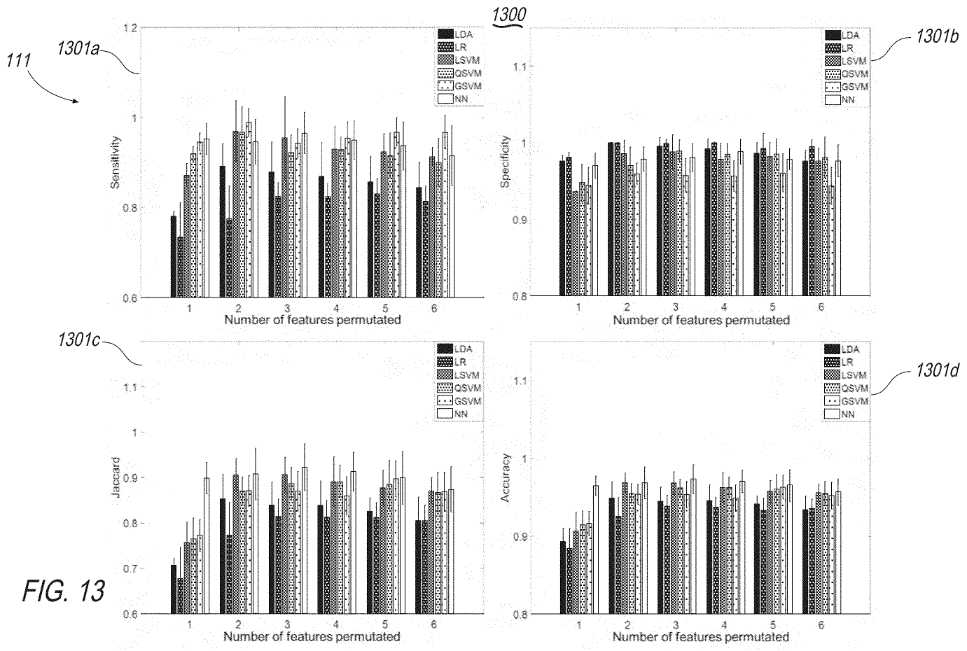

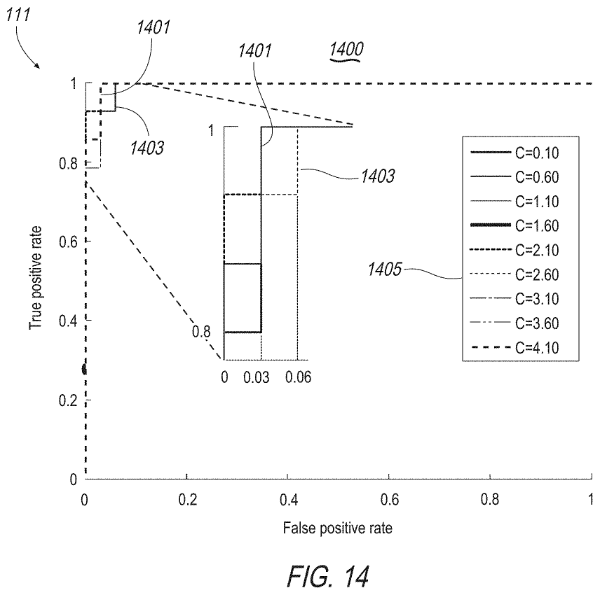

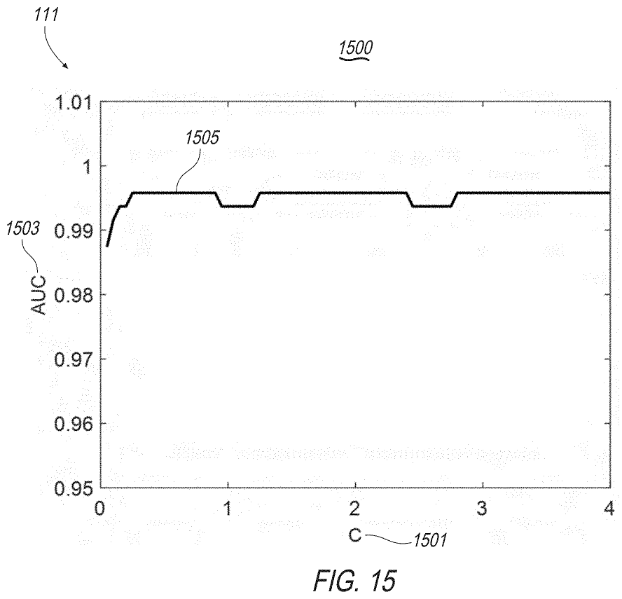

FIGS. 13-15 illustrate optical information generated by processor 105 for display on display 111. FIG. 13 illustrates optical information 1301a,b,c,d of the best results for each classifier. FIG. 14 illustrates optical information 1401 including a receiver operating characteristic (ROC) curve for GSVM classifier produced by changing the margin factor, e.g., C, from 0 to 4 with steps 0.1 FIG. 15 illustrates optical information including the area under the curve (AUC) for each margin.

System 100 may generate, store and display optical information as set forth in Table 3 below including the best sensitivity, specificity, Jaccard index and accuracy for combinations of four features.

TABLE 3

| Feature | | | | | |
|---|---|---|---|---|---|
| combination | | | | Jaccard | |
| [F₁F₂F₃F₄F₅F₆] | Classifier | Sensitivity | Specificity | Index | Accuracy |
| [010111] | LDA | 0.87 ± 0.07 | 0.96 ± 0.03 | 0.8 ± 0.07 | 0.93 ± 0.03 |
| [011110] | LDA | 0.82 ± 0.05 | 0.99 ± 0.01 | 0.81 ± 0.05 | 0.94 ± 0.02 |
| [101110] | LDA | 0.86 ± 0.04 | 0.99 ± 0.02 | 0.84 ± 0.05 | 0.94 ± 0.02 |
| [101101] | LR | 0.82 ± 0.03 | 0.99 ± 0.01 | 0.81 ± 0.04 | 0.94 ± 0.01 |
| [011110] | LR | 0.81 ± 0.04 | 1.0 ± 0.0 | 0.81 ± 0.04 | 0.94 ± 0.01 |
| [111100] | LSVM | 0.93 ± 0.05 | 0.98 ± 0.01 | 0.89 ± 0.05 | 0.96 ± 0.02 |
| [001111] | LSVM | 0.90 ± 0.03 | 0.98 ± 0.02 | 0.86 ± 0.04 | 0.95 ± 0.01 |
| [111100] | QSVM | 0.93 ± 0.03 | 0.98 ± 0.02 | 0.89 ± 0.04 | 0.96 ± 0.01 |
| [011101] | QSVM | 0.87 ± 0.07 | 0.99 ± 0.01 | 0.85 ± 0.07 | 0.95 ± 0.03 |
| [101110] | GSVM | 0.95 ± 0.04 | 0.94 ± 0.03 | 0.85 ± 0.05 | 0.95 ± 0.02 |
| [110110] | GSVM | 0.91 ± 0.04 | 0.96 ± 0.02 | 0.84 ± 0.05 | 0.94 ± 0.02 |
| [101101] | GSVM | 0.95 ± 0.04 | 0.95 ± 0.03 | 0.86 ± 0.04 | 0.95 ± 0.02 |
| [101101] | NN | 0.95 ± 0.04 | 0.98 ± 0.02 | 0.91 ± 0.04 | 0.97 ± 0.01 |
| [110101] | NN | 0.92 ± 0.06 | 0.99 ± 0.02 | 0.90 ± 0.05 | 0.97 ± 0.02 |

System 100 may generate, store and display optical information including the best sensitivity value or range, e.g., Jaccard index and accuracy for combinations of four features. Table 4 below shows the optimum selection of classifier and feature combinations for sensitivity, specificity, Jaccard index and accuracy and combinations thereof. The best overall may be a combination of features 1 through 5 with the GSVM classifier (C=2.1). Optimum selection of classifier and feature combinations to achieve the optimum sensitivity, specificity, Jaccard index and accuracy, individually; the best sensitivity (row 1); the best specificity (row 2); the best Jaccard index (row 3); the best accuracy (row 4); statistical results when GSVM with a margin factor of 1 (row 5) and 2.1 (row 6) was used. The binary numbers in “Feature combination” column show if that feature has been used, “1” or not, “0”.

TABLE 4

| | Feature | | | | | |
|---|---|---|---|---|---|---|
| | combination | | | | Jaccard | |
| Row | [F₁F₂F₃F₄F₅F₆] | Classifier | Sensitivity | Specificity | Index | Accuracy |
| 1 | [011000] | GSVM | 0.99 ± 0.03 | 0.50 ± 0.01 | 0.49 ± 0.01 | 0.66 ± 0.01 |
| | | (C = 1) | | | | |
| 2 | [001100] | LDA | 0.81 ± 0.05 | 1 ± 0.0 | 0.81 ± 0.05 | 0.94 ± 0.02 |
| 3 | [110100] | NN | 0.97 ± 0.05 | 0.98 ± 0.02 | 0.92 ± 0.05 | 0.97 ± 0.02 |
| 4 | [110100] | NN | 0.97 ± 0.05 | 0.98 ± 0.02 | 0.92 ± 0.05 | 0.97 ± 0.02 |
| 5 | [111110] | GSVM | 0.97 ± 0.03 | 0.96 ± 0.03 | 0.90 ± 0.04 | 0.96 ± 0.02 |
| | | (C = 1) | | | | |
| 6 | [111110] | GSVM | 0.97 ± 0.03 | 0.98 ± 0.02 | 0.93 ± 0.05 | 0.98 ± 0.02 |
| | | (C = 2.1) | | | | |

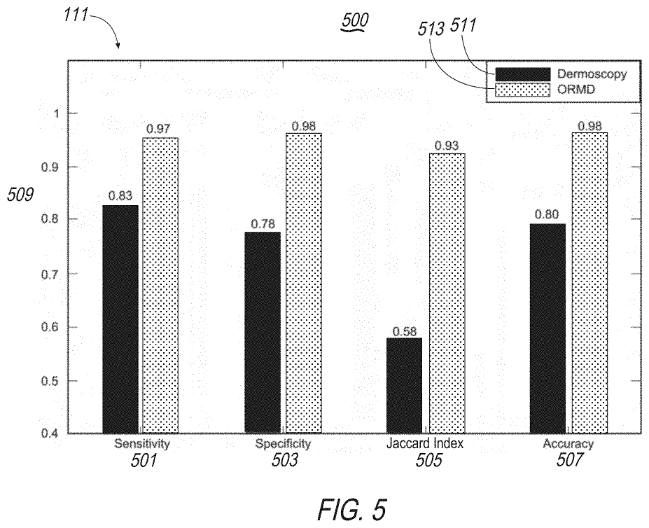

As shown in FIG. 5, system 100 may generate, store, transfer and display screen 500 on display 111, e.g., including optical information such as a comparison of diagnostic statistics based on dermoscopic and optical radiomic melanoma detection (ORMD) criteria for the selected optimum classifier (GSVM classifier (C=2.1)) and optimum feature set. For example, this may include sensitivity 501, specificity 503, Jaccard index 505 and accuracy 507 of melanoma detection based on dermoscopic criteria and ORMD criteria. The optimum classifier (e.g., GSVM classifier (C=2.1)) may be used with the optical radiomic signatures, including, mean and standard deviation of scattering and absorption coefficients, and the mean of the anisotropy factor.

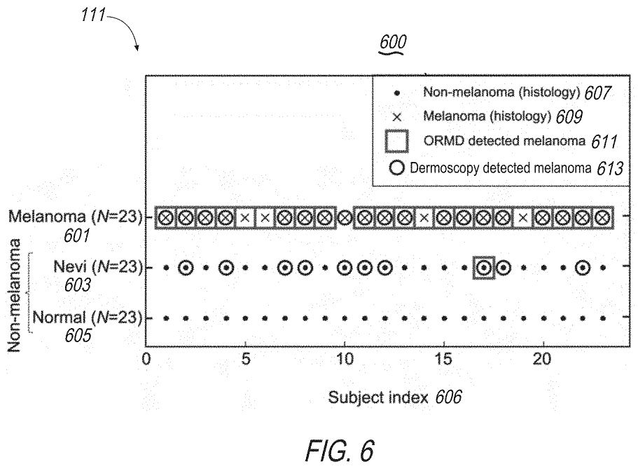

Referring to FIG. 6, system 100 may generate, store, transfer and display screen 600 on display 111, e.g., including optical information with classification results. This may include a comparison of dermoscopic diagnosis, ORMD method, and histology. Benign (healthy skin and benign nevi) are marked as dots while melanomas are marked with crosses. The tissue statuses may be confirmed by histological analysis. System 100 may include a first identifier having a first color and a first shape (e.g., blue circles) to indicate detection of melanoma using dermoscopy. System 100 may include a second identifier having a second color and a second shape (e.g., red squares) indicating detection of melanoma using the ORMD method. GSVM classifier with the margin factor of 2.1 may be used. The system 100 may generate and display one or more outputs. For example, the outputs may include a singular, binary or multi-factor output such as (1) “Tissue sample exhibits characteristics consistent with melanoma”, the lesion should be considered for biopsy; or (0) “Tissue sample is consistent with healthy tissue”, the lesion does not require biopsy. This may also include a comparison of dermoscopic diagnosis, ORMD method, and histology. Non-melanoma (healthy skin and benign nevi) are marked as dots while melanomas are marked with crosses. The tissue statuses may be confirmed by histological analysis. A first indictor including a first color and/or a first shape (e.g., blue circles) may indicate detection of melanoma using dermoscopy. A second indicator including a second color and/or a second shape (e.g., red squares) may include detection of melanoma using the ORMD method. System 100 may utilize a GSVM classifier with the margin factor of 2.1.

System 100 may include optical information include an image analysis method to disaggregate OCT images into individual optical attributes. System 100 may utilize EHF principles, referred to as OPE. These optical attributes, when extracted from the OCT image form a set of tissue specific optical radiomic features. The systems and methods herein provide improvements in melanoma detection over traditional clinical methods. Initial tests may be conducted by system 100 on milk and milk-ink phantoms. System 100 may determine if the OPE method correctly correlates to changes in optical properties of the phantoms, e.g., scattering and absorption coefficients. See FIG. 3.

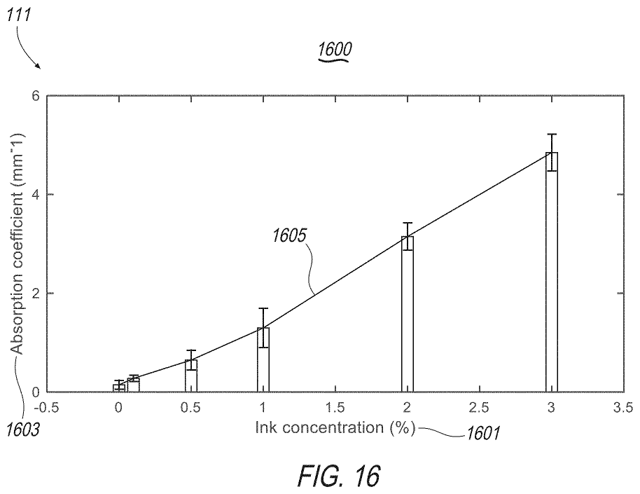

System 100 may generate, store and display optical information including OPE-extracted optical properties. This may include the scattering coefficient (μₛ) progressed almost linearly with increasing milk concentration (p<0.001); the absorption coefficient (μₐ) in milk phantoms progressed almost linearly with increasing the milk concentration (p<0.001); the absorption coefficient in milk-ink phantoms progressed almost linearly with increasing the ink concentration (p<0.01). The results in FIG. 2(g) appear nonlinear because of nonlinear scaling of x-axis. The linearized plot is shown in FIG. 16. System 100 may increase both the absorption and scattering coefficients by increasing the concentration of milk, but this does not indicate cross-talk between the scattering and absorption coefficients but indicates the presence of both scattering and absorption properties in milk as both are accurately extracted using the OPE method.

Optical information may also include an OPE-extracted μₛ in milk-ink phantoms shows no statistically significant difference (p<0.001 with λ=1 [mm⁻¹]). The values of anisotropy factor (g) also show no statistically significant difference in both milk and milk-ink phantoms (p<0.05 with Δ=0.03), which is consistent with the phantoms being homogenous solutions consisting of scatterers of near identical size. Different values of delta may be chosen for different settings and the values may be based on preliminary results for clinical importance. The average fitting error in both datasets may be about 4%. Precision of the obtained values can be improved by using a higher resolution OCT.

System 100 may include a non-invasive, OCT system with IRB approval for humans. See FIG. 1. Sixty-nine melanoma, benign nevi and healthy subjects may be recruited. See FIGS. 20A, 20B, and 20C. The results obtained from the clinically identified melanoma to benign area showed a meaningful difference. See FIG. 3. Differences due to factors such as skin type, ethnicity, sun exposure, etc., may be negated when normalized to nearby healthy skin. The large standard deviation of the optical radiomic features for melanoma images correlates to irregularity in tissue structure; signifying disease. The results may be consistent with the finding that the scattering and absorption coefficients increase with the concentration of melanocytes (melanocyte frequency—melanoma: 71±11%; benign nevi: 18±3%; healthy: 14±3%); anisotropy factor increased with cell size (average mean diameter of 200 consecutive melanocytes—melanoma: 16±3 μm; benign nevi: 7±0.4 μm; healthy: 6±0.4 μm) and tissue disorder, due to cellular displacement. System 100 may include an OMLC generator for various simulations. See Tables 5-7.

Increases in scattering and absorption coefficients may be due to increased concentration of melanocytes, and the increase in anisotropy factor may be due to increased cell size. See FIG. 3. The combination of increased numbers of melanocytes that are larger with pleomorphic nuclei is the hallmark of melanoma on pathological assessment.

System 100 may generate a predefined number (e.g., six) of optical radiomic features from the OCT images. This may include the mean and standard deviation of scattering coefficient, absorption coefficient and anisotropy factor. With the predefined number of features, each possible combination of features may be examined to identify the optimal feature set. This search reaches the optimal feature sets by systematically enumerates all possible candidates. System 100 identifies an optimal feature set more efficiently than other feature selection methods such as sequential floating forward search (SFFS) and sequential floating backward search (SFBS).

As for the criteria to choose the most appropriate classifier, a true class probability density function (pdf) is estimated. With small to medium size datasets, such a function may be difficult to accurately estimate, and the performance of the classifiers is difficult to calculate. As a rule of thumb, low variance classifiers (e.g., Naïve Bayes, SVM) are preferred for such datasets. The disclosed method is to find the best classifier with the aid of validation/training and a repeated random sampling strategy. Six established classifiers may be selected, each may be trained and tested on the data using a 20-fold cross validation process; and this evaluates the classifier generalization. Values for sensitivity, specificity, Jaccard index and accuracy, may be determined by testing permutations of the six features, in combination with each classifier. See FIG. 13 and Tables 3 and 4.

Based on clinical requirements of high specificity and sensitivity, a specific classifier and set of features may be selected. Some combinations generated high sensitivity with low specificity, or vice versa. For example, features F₂, F₃, with the GSVM (C=1) classifier resulted in the best sensitivity (99%) with a specificity of 50% (for more examples, see Table 4).

The best overall may be a combination of features F₁ through F₅ with the GSVM classifier, results may be sensitivity (97% 3%), specificity (98% 2%), Jaccard index (93%±5%), and accuracy (98% 2%) (see FIG. 4). For the preferred classifier, GSVM, the area under the curve (AUC) may be calculated with different C-values, and C=2.1 gave us the best results. See FIGS. 14-10.

System 100 may perform a dermoscopic analysis may be made using a one-, two- or multi-step assessment followed by pattern analysis of optical information. The suspicious lesions may be selected based on changes on dermoscopic follow-up. Assessment of dermoscopy images compared to results of the ORMD methodology, showed a significant diagnostic improvement. See FIG. 5. Using ORMD only one unnecessary biopsy for melanoma may be performed, while dermoscopy identified 10 benign nevi as possible melanoma, necessitating 10 biopsies. In melanoma, OPE missed one case, where dermoscopy misdiagnosed four cases as benign nevi, resulting in delayed treatment.

System 100 may generate statistics indicating that ORMD-based diagnosis is reliable and can effectively differentiate between melanoma and benign cases (see FIGS. 4 and 5), a larger number of subjects makes a more rigorous conclusion. Overall, the rate of unnecessary biopsies is significantly decreased with the use of the ORMD methodology. A larger number of subjects may necessitate the use of a more sophisticated classification process which may further increase the accuracy of the ORMD methodology and minimize the number of misdiagnoses.

Thus, according to the disclosure, OCT images from suspect lesion and nearby healthy skin are the inputs to the OPE method, which is the core of the disclosed ORMD method. As described, a precise physical OCT model is used in the disclosed OPE method to extract the optical properties of a tissue from a specific region of interest.

Referring again to FIG. 6, system 100 may generate and display screen 600 including optical information such as diagnostic results of operations of the OPE method. A region of interest is specified in an OCT B-scan image. The pixel intensities along the x-axis in each ROI are averaged to obtain an averaged A-line. Using a fitting algorithm, the scattering and absorption coefficients as well as the anisotropy factor in the modeled OCT signal are adjusted, in order to obtain a curve that best fits the averaged A-line. By repetition for several regions of interest (ROIs), which are averaged, and standard deviations calculated, optical radiomic features can be derived for that tissue: mean and standard deviation of scattering and absorption coefficients, and anisotropy factor. These radiomic features obtained from the suspect lesion and its nearby healthy skin are used to create a set of normalized optical radiomic features, that accounts for gender, age and skin color.

With reference again to FIG. 1, system 100 may include a computing device 103 having a processor 105, display 111 and memory 107 including a program 105 to perform any of the operations herein. For example, the system 100 may compare inputs and outputs, identify and classify patterns between the inputs and outputs, and automatically adapt any of the same to optimize the results, with or without human intervention. System 100, e.g., program 109, may include heuristics to provide, for example, machine learning, artificial intelligence, deep learning, deep neural learning and/or deep neural network. The system 100 may provide operations for receiving and processing inputs (e.g., data), creating comparisons (e.g., patterns), creating and refining the operations herein (e.g., supervised, unsupervised or automated learning), and providing, transferring and/or displaying optical information including outputs (e.g., optimized results or unstructured or unlabeled data).

System 100 may include heuristics (e.g., machine learning) configured for supervised, unsupervised or automated classification for training between optical radiomic features of melanoma and benign to train system 100, and to provide for automatic adaptation based on correlations between inputs, outputs and diagnostic results for improved accuracy across a wider variation of melanoma types and stages. System 100 may compare and validate inputs, outputs and diagnostic results histologically with nuanced variations in cytology to train and automatically adapt the heuristics to identify melanoma from its earliest instance. System 100 may utilize a priori knowledge of OCT images, and of healthy and melanoma tissue histology, to train the heuristics (e.g., machine-learning kernel) with improved decision-making over a traditional system using statistical classifiers.

The heuristics may include one or multiple phases including, for example, (i) a Training Phase, and (ii) a Test Phase. In the Training Phase, the optical radiomic features and their labels (histology results) are input to the heuristics. In the Test Phase, OCT images of a suspect skin area will be analyzed by the trained heuristics (e.g., machine-learning kernel) with the selected optical radiomic features, e.g., optical radiomic signatures. The system 100 may display the status of the tissue associated with healthy or benign tissue, e.g., “Tissue sample is consistent with healthy tissue” or cancerous or melanoma tissue, e.g., “Tissue sample exhibits characteristics consistent with melanoma”.

Referring again to FIG. 2, principles of ORMD algorithm. μₛ: scattering coefficient, μₐ: absorption coefficient, g: anisotropy factor. System 100 may include preprocessing operations.

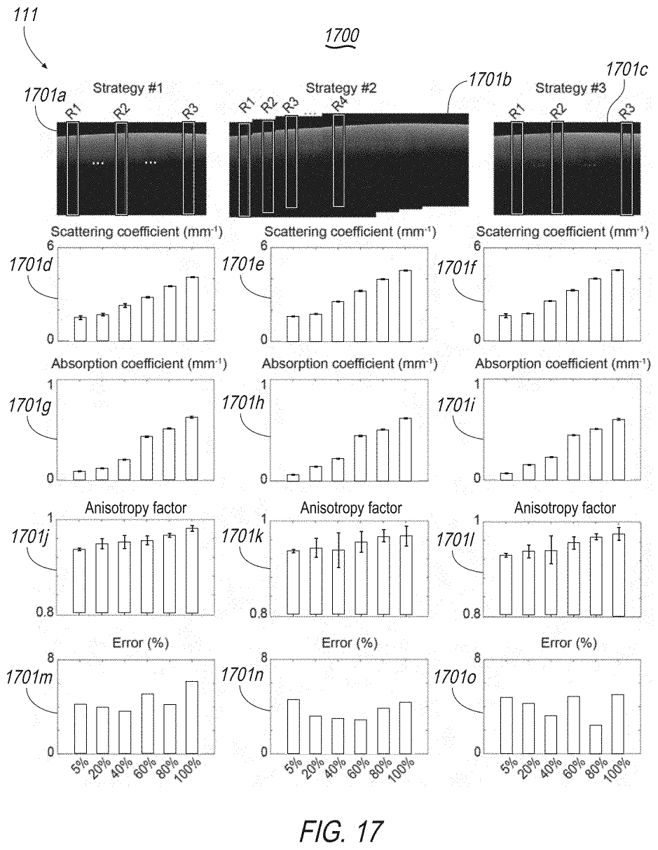

One of the main steps in the optical properties extraction (OPE) method is choosing an appropriate size for the region of interest (ROI), for which the optical properties are calculated. Different ways of choosing the ROI may be investigated on optical coherence tomography (OCT) images of milk phantoms: (1) a median filter may be initially applied on a stack of 170 OCT images acquired from the same cross section, the extracted optical properties may be averaged over several ROIs chosen in the resultant image; (2) the same ROI may be chosen in 170 images and the extracted optical properties may be averaged; (3) a single B-scan may be randomly selected from 170 images and the optical properties may be extracted from several ROIs and averaged; for strategies (1) and (3), 24 ROIs may be considered in the OCT image, each included 100 A-scans. Running equivalence test on the results, the statistical difference between the optical properties obtained from strategy #3 with those from strategies #1 and #2 may be insignificant (p<0.05 with Δit of the scattering coefficient=0.5 [mm⁻¹], λ of the absorption coefficient=0.03 [mm⁻¹], and A of the anisotropy factor=0.01), therefore the optical properties extracted from a single B-scan image can be as accurate as the ones extracted from the average of several images (see FIG. 17). Since the OPE methodology will be used on OCT images of skin, due to the layered structure of the skin tissue, an optimum size of the ROI needs to be determined to generate robust results.

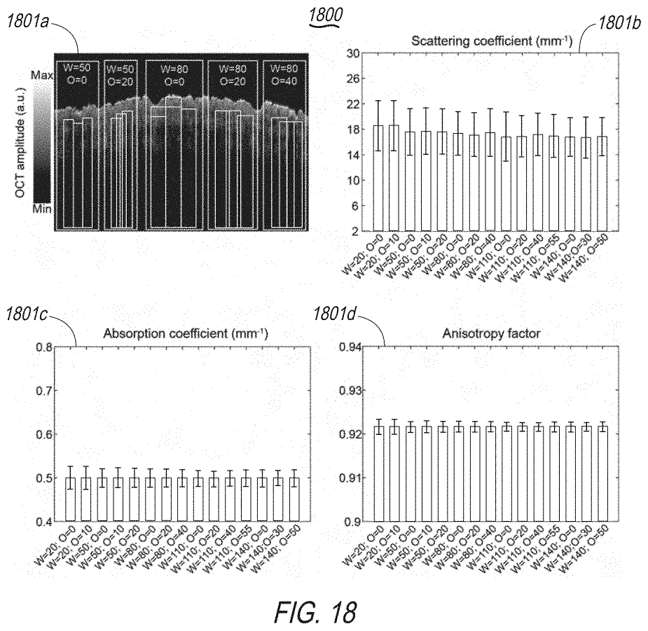

For example, a stack of 60 OCT images may be acquired from different transverse cross-sections of the forearm of a 30-year-old male who had no skin condition. The variation of the scattering coefficient, absorption coefficient and anisotropy factor with different sized ROIs, when they may be overlapped, and with different overlap spans may be explored. Initially, ROIs with different widths 20, 50, 80, 110, and 140 pixels (89, 223, 357, 490, and 624 μm), and with overlap widths of 10, 20, 40 and 50 pixels may be tested. The results in FIG. 18 shows that the optical properties obtained from these conditions are similar (p<0.05 with λ=1 [mm⁻¹] for scattering coefficient, λ=0.05 [mm⁻¹] for absorption coefficient, and λ=0.01 for anisotropy factor). The analysis suggests that the OPE method generates statistically similar results in different size ROIs in a single OCT image. Considering a slight difference between the results, the optimum width for the ROI is 80 pixels with an overlap of 20 pixels. To optimize the length of the ROI, ROIs with varying lengths may be considered. The best length of the ROI may be obtained 180 pixels based on two considerations: i) a few number of pixels cannot provide a sufficient number of samples for fitting, ii) considering low signal-to-noise ratio (SNR) pixels in fitting process generates a larger error. In total, 24 ROIs may be selected in each image. The average and standard deviation of optical properties over all ROIs of the image are calculated and reported as mean and standard deviation of the optical properties of that image.

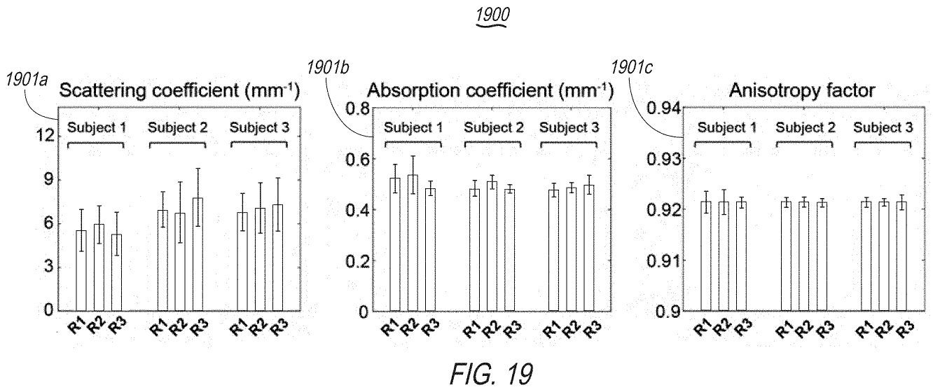

An optimum ROI size may be obtained from the previous experiments to compare the optical properties of the skin of the forearm of three healthy individuals. The subjects chosen for this experiment may be 25 and 30-year-old males and a 30-year-old female, none of whom had any skin conditions. 3 regions (R1, R2 and R3) may be imaged on each subject's forearm with a 10 mm distance between them. The images may be collected from 6 mm by 6 mm transverse areas. The average and standard deviation of the scattering coefficients, absorption coefficients, and anisotropy factor for each subject for the R1, R2, and R3 may be compared. The results indicated an insignificant difference between the optical properties extracted from the same individual (see FIG. 19). This may be to make sure that the difference between the optical properties extracted from adjacent regions may be statistically insignificant. An equivalence test may be performed between every pair of regions in each subject and resulted p<0.001 with λ=1 [mm⁻¹] for scattering coefficient, p<0.05 with λ=0.1 [mm⁻¹] for absorption coefficient, and p<0.001 with λ=0.01 for anisotropy factor. In this test, the null hypothesis may be the absolute difference between the average of two experimental settings (e.g., |mean_{A}−mean_{B}|) is higher than a threshold value, λ. Different values of delta may be chosen for different settings and the values may be based on our preliminary results for clinical importance. The rejection of the null hypothesis indicates the equivalence of the two conditions. All the other statistical tests may be two sided at the 5% level of significance

System 100 may include Mie simulations. When light interacts with a spherical particle with geometrical cross-section area A [L²], an effective scattering cross-section, σₛ [L²], is calculated as σₛ=Qₛ×A, where Qₛ [dimensionless] is the scattering efficiency. For a volume where many such particles are homogeneously distributed, the scattering coefficient is defined as μₛ=σₛ×ρₛ, where ρₛ represents the density of particles per volume [L⁻³] and μₛ has a unit of [L⁻¹]. The scattering coefficient can also be thought of as the reciprocal of the average distance a photon travels between scattering events. Note that while the scattering cross-section, σₛ, is a microscopic property of a particle, the scattering coefficient, μₛ, is a macroscopic average of a medium. Analogous to the scattering coefficient, for the absorption coefficient an effective absorption cross-section σₐ [L²] is calculated which is related to the geometrical cross-section by the absorption efficiency Qₐ [dimensionless]. Likewise, in the macroscopic case, the absorption coefficient, μₐ [L⁻¹], can be defined as μₐ=σₐ×ρₐ, where ρₐ is the density of absorbers in the medium[L⁻³]. Following these relationships, therefore, there is a direct relation between μₛ and μₐ with the density of scatterers/absorbers in a volume, which explains why the scattering coefficient and absorption coefficients increase with the concentration of scatterers and absorbers (e.g., melanocytes in the skin tissue). To demonstrate this, a simulation may be performed using Mie theory principles and using online Mie calculator, which works based on solving Maxwell's equations for the interaction of light with tissue. The input to the simulator may be as follows: scatterer structure may be simplified and considered as a sphere, central wavelength of the OCT light source may be set to 1305 nm and the refractive index of scatterers as 1.3, the average refractive index of skin. In Tables 5-7, the scattering and absorption coefficients, as well as the anisotropy factors are reported.

System 100 may compare by processor 105 and display by display 111 a comparison of the concentration of scatterers and their scattering coefficients.

TABLE 5

| Concentration | | Scattering | |
|---|---|---|---|
| (spheres per | Cell size | coefficient | Anisotropy |
| cubic micron) | (micron) | (mm⁻¹) | factor |
| 0.0001 | 6 | 5.8555 | 0.74169 |
| 0.0002 | 6 | 11.711 | 0.74169 |
| 0.0003 | 6 | 17.566 | 0.74169 |

System 100 compare by processor 105 and display by display 111 a comparison of the particle size and their anisotropy factor.

TABLE 6

| Concentration | | Scattering | |
|---|---|---|---|
| (spheres per | Cell size | coefficient | Anisotropy |
| cubic micron) | (micron) | (mm⁻¹) | factor |
| 0.0001 | 6 | 5.8555 | 0.74169 |
| 0.0001 | 16 | 48.095 | 0.85191 |
| 0.0001 | 26 | 114.82 | 0.85669 |

System 100 may compare by processor 105 and display by display 111 a comparison of the concentration of absorbers and absorption coefficient as set forth in Table 7 below.

TABLE 7

| Concentration | | Absorption |
|---|---|---|
| (spheres per | Cell size | coefficient |
| cubic micron) | (micron) | (mm⁻¹) |
| 0.0001 | 6 | 0.0123 |
| 0.0002 | 6 | 0.0246 |
| 0.0003 | 6 | 0.0369 |

System 100 may generate, store, display and transfer optical information including inputs and outputs indicating diagnostic results. FIG. 7 illustrates histologic photographs and OCT images of five selected melanoma cases and five nevus cases, which may include including datasets for normal and abnormal parts and/or a scale bar in the histology images and the OCT images (e.g., 1 mm). FIG. 8 includes a pre-processing procedure optimization. FIG. 9 illustrates scattering coefficients, absorption coefficients, and anisotropy factors of five melanoma (“2”) cases and their nearby healthy (“1”) skin (calculated from 3 consecutive OCT slices). FIG. 10 includes scattering coefficients, absorption coefficients, and anisotropy factors of five benign nevi (“2”) cases and their nearby normal (“1”) skins (calculated from 3 consecutive OCT slices). FIG. 11 includes scattering coefficients, absorption coefficients and anisotropy factors of melanoma cases and their nearby normal skin for the remaining 18 cases (apart from or in addition to those in FIG. 3) such as Scattering coefficients 1100a, absorption coefficients 1101b, and anisotropy factor 1101c. FIG. 12 includes scattering coefficients, absorption coefficients and anisotropy factors of benign nevi cases and their nearby normal skin for the remaining 18 cases (apart from or in addition to FIG. 3) including scattering coefficients 1201a, absorption coefficients 1201b, and anisotropy factor 1201c.

System 100 may generate and display optical information including, for example, a patient-specific and/or optimized diagnosis. FIG. 13 includes classifier and feature selection optimization including the best of any or all of: sensitivity 1301a, specificity 1301b, Jaccard index 1301c, and accuracy 1301d, e.g., for various feature combinations using each classifier. System 100 may include and utilize linear discriminant analysis (LDA), linear regression (LR), linear support vector machine (LSVM), quadratic support vector machine (QSVM), Gaussian support vector machine (GSVM), nearest neighbor (NN), or a combination thereof.

System 100 may generate and display optical information including, e.g., diagnosis results. FIG. 14 illustrates a ROC curve for sample margin factors in GSVM. Margin factors from 0 to 4 with steps 0.1 have been evaluated. System 100 may include and utilize a receiver operating characteristic (ROC), Gaussian support vector machine (GSVM), or a combination thereof.

FIG. 15 illustrates an area under the curve (AUC) for selected margin factors when GSVM classifier may be used. FIG. 16 illustrates absorption coefficients with linearized X-axis, e.g., based on optical information 301g of FIG. 3. The concentration of milk in all these experiments may be 5%.

System 100 may execute and display optical information using pre-processing operations. FIG. 17 illustrates three pre-processing methodologies using by a series of OCT images of the milk phantom. X-axis shows the concentration of milk diluted by water. System 100 may include screen 1700 including optical information 1701. Optical information 1701a may include smoothed OCT image on which several ROIs are specified, optical information 1701b may include an ROI located at the same place in all the images collected from the same place in the sample, optical information 1701c may include an OCT image of the milk phantom on which several ROIs are specified, optical information 1701d,g,j,m may include scattering coefficient, absorption coefficient, anisotropy factor and corresponding fitting error for a first preprocessing method, optical information 1701e,h,k,n may include scattering coefficient, absorption coefficient, anisotropy factor and corresponding fitting error for a second preprocessing method, optical information 1701f,i,l,o may include scattering coefficient, absorption coefficient, anisotropy factor and corresponding fitting error for a third preprocessing strategy. The equivalence test resulted p<0.05 for all corresponding pairs of optical properties for each of the first, second and third preprocessing methods.

System 100 may generate and display the ROI with an optimized size. FIG. 18 illustrates screen 1800 including optical information 1801. Optical information 1801a may include an OCT image of a dorsal hand sample on which five example scenarios are depicted in each white box, optical information 1801b may include scattering coefficients, optical information 1801c may include absorption coefficients, and optical information 1801d may include anisotropy factor of ROIs of various widths (W) and overlaps (O). FIG. 19 illustrates screen 1900 including optical information 1901a including scattering coefficients, optical information 1901b including absorption coefficients, and optical information 1901c including anisotropy factors, e.g., of three adjacent regions (R1, R2 and R3) on the forearm of three subjects (e.g., 10 mm distances).

When introducing elements of various embodiments of the disclosed materials, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While the preceding discussion is generally provided in the context of medical imaging, it should be appreciated that the present techniques are not limited to such medical contexts. The provision of examples and explanations in such a medical context is to facilitate explanation by providing instances of implementations and applications. The disclosed approaches may also be utilized in other contexts, such as the non-destructive inspection of manufactured parts or goods (e.g., quality control or quality review applications), and/or the non-invasive inspection or imaging techniques.

While the disclosed materials have been described in detail in connection with only a limited number of embodiments, it should be readily understood that the embodiments are not limited to such disclosed embodiments. Rather, that disclosed can be modified to incorporate any number of variations, alterations, substitutions or equivalent arrangements not heretofore described, but which are commensurate with the spirit and scope of the disclosed materials. Additionally, while various embodiments have been described, it is to be understood that disclosed aspects may include only some of the described embodiments. Accordingly, that disclosed is not to be seen as limited by the foregoing description, but is only limited by the scope of the appended claims.

What is claimed is:

1. A system for using optical coherence tomography (OCT) to detect melanoma, comprising:
a scanning probe configured to image skin; and
a computing device having a hardware processor and physical memory, and communicatively connected to the scanning probe to provide operations including:
obtain a first image of a suspect region-of-interest (SROI) for a suspect lesion;
obtain a second image of a healthy region-of-interest (HROI) near the suspect lesion;
classify the extracted optical properties to generate a tissue status including as at least one of a melanoma tissue and a benign tissue; and
display the tissue status indicating the at least one of the melanoma tissue and the benign tissue.

2. The system of claim 1, the operations further including normalize optical properties from the SROI and from the HROI, and obtain an averaged A-line of the SROI and the HROI.

3. The system of claim 1, the operations further including generate a set of normalized optical radiomic features from an averaged A-line of the SROI and the HROI.

4. The system of claim 1, the classify operation including evaluate the set of normalized optical radiomic features to distinguish whether the suspect lesion is consistent with the at least one of the melanoma tissue and the benign tissue.

5. The system of claim 1, wherein the first and second images are B-scans.

6. The system of claim 1, wherein the first and second images are at least one of A-scans, B-scans, C-scans, Fourier-domain (FD) scans, spectral-domain (SD) scans, and three-dimensional (3D) scans.

7. The system of claim 1, the operations further comprising display optical information including at least one of optical properties, normalized optical properties, and classified optical properties indicating the at least one of the melanoma tissue and the benign tissue.

8. A device for using optical coherence tomography (OCT) to detect melanoma, having a hardware processor and physical memory, and communicatively connected to the scanning probe to provide operations comprising:
obtain a first image of a suspect region-of-interest (SROI) for a suspect lesion;
obtain a second image of a healthy region-of-interest (HROI) near the suspect lesion;
extract optical properties from the SROI and from the HROI;
classify the extracted optical properties to generate an issue status including as at least one of a melanoma tissue and a benign tissue; and
display the tissue status indicating the at least one of the melanoma tissue and the benign tissue.

9. The device of claim 8, the operations further including normalize optical properties from the SROI and from the HROI, and obtain an averaged A-line of the SROI and the HROI.

10. The device of claim 1, the operations further including generate a set of normalized optical radiomic features from an averaged A-line of the SROI and the HROI.

11. The device of claim 1, the classify operation including evaluate the set of normalized optical radiomic features to distinguish whether the suspect lesion is consistent with the melanoma tissue and the benign tissue.

12. The device of claim 1, wherein the first and second images are B-scans.

13. The device of claim 1, wherein the first and second images are at least one of A-scans, B-scans, C-scans, Fourier-domain (FD) scans, spectral-domain (SD) scans, and three-dimensional (3D) scans.

14. The device of claim 1, the operations further comprising displaying optical information including at least one of optical properties, normalized optical properties, and classified optical properties indicating the at least one of the melanoma tissue and the benign tissue.

15. A method of using optical coherence tomography (OCT) to detect melanoma, comprising:
providing a computing device having a hardware processor and physical memory;
communicatively connecting the computing device to a scanning probe:
obtaining a first image of a suspect region-of-interest (SROI) for a suspect lesion;
obtaining a second image of a healthy region-of-interest (HROI) near the suspect lesion;
extracting optical properties from the SROI and from the HROI;
classifying the extracted optical properties to generate a tissue status including as at least one of a melanoma tissue and a benign tissue; and
displaying the tissue status indicating the at least one of the melanoma tissue and the benign tissue.

16. The method of claim 15, the operations further including normalizing optical properties from the SROI and from the HROI, and obtain an averaged A-line of the SROI and the HROI.

17. The method of claim 15, the operations further including generating a set of normalized optical radiomic features from an averaged A-line of the SROI and the HROI.

18. The method of claim 15, the classify operation including evaluating the set of normalized optical radiomic features to distinguish whether the suspect lesion is consistent with the melanoma tissue and the benign tissue.

19. The method of claim 15, wherein the first and second images are B-scans.

20. The method of claim 1, wherein the first and second images are at least one of A-scans, B-scans, C-scans, Fourier-domain (FD) scans, spectral-domain (SD) scans, and three-dimensional (3D) scans.
